(19) United States
(12) Patent Application Publication
(10) Pub. No.: US 2020/0311056 A1
(43) Pub. Date: Oct. 1, 2020

(54) METHOD AND SYSTEM FOR SUBSTITUTING A FOOD OR DRINK OR ONE OR MORE INGREDIENTS THEREOF

(71) Applicant: FOODPAIRING NV, Ghent (BE)

(72) Inventors: Dries ROBBERECHTS, Wondelgem (BE); Bernard LAHOUSSE, Kortrijk (BE); Peter COUCQUYT, Deerlijk (BE); Johan LANGENBICK, Aalter (BE); Hendrik D'OOSTERLINCK, Zonnegem (BE)

(21) Appl. No.: 16/590,314

(22) Filed: Oct. 1, 2019

Related U.S. Application Data
(63) Continuation of application No. 14/607,033, filed on Jan. 27, 2015, now abandoned.
Application No. 14/607,033 is a continuation-in-part of application No. 14/165,455, filed on Jan. 27, 2014, now Pat. No. 10,162,481.

(51) Int. Cl. G06F 16/24; G06F 3/0482; G06F 3/0484; G09B 19/00; G16H 20/60

(52) U.S. Cl. CPC G06F 16/24; G06F 3/0482; G16H 20/60; G09B 19/0092; G06F 3/04842

(57) ABSTRACT

Disclosed herein are methods and systems for substituting an existing ingredient or dish with alternative ingredients and dishes for food and drinks. In particular, the substitution of ingredient or dish is achieved based on sensory properties of an ingredient or dish such as taste, aroma, texture and etc. Also disclosed herein are computer-implemented methods and systems for performing the ingredient or dish substitution.

### CROSS-REFERENCE TO RELATED APPLICATIONS

This application claims priority from U.S. patent application Ser. No. 14/165,455, filed on Jan. 27, 2014 and entitled “METHOD AND SYSTEM FOR CREATING A FOOD OR DRINK RECIPE,” which is incorporated herein by reference in its entirety.

### FIELD

The invention disclosed herein generally relates to alternative ingredients and dishes for food and drinks. The invention discloses herein relates to the generation and presentation of sensory properties of an ingredient or dish such as taste, aroma, texture and etc.; and the generation of ingredient and dish alternatives based on its sensory properties. Furthermore, the present invention relates to a method and system for substituting a dish (e.g., a food or drink product) or one or more ingredients thereof by one or a combination of multiple other ingredients or dishes, based on physicochemical data of those ingredients or dishes. Still further, the present invention relates to computer-based methods and systems for implementing such substitution of ingredient or dish.

### BACKGROUND

When searching, shopping, or preparing a food or drink based on a recipe including multiple ingredients, many situations occur where one or more alternative ingredients for the recipe would be advantageous. In some cases, it is even desirable to have alternative recipes at hand to create alternative and comparable dishes. Exemplary situations include but are not limited to the following: a) a desired ingredient is not available; b) because of diet restrictions, medical conditions, and food allergies, a certain ingredient cannot be consumed; c) an ingredient from the recipe is not liked; d) cost of certain ingredient is too high; or e) the impact on the environment of a certain ingredient is too high. In other scenarios, a person can be motivated to add variations in the menu or an urge to know or experiment with other/new similar ingredients and experience sensorial variation.

Replacement or substitution of one or more ingredient or a dish (e.g., a food or drink product) offers many benefits. For example, substitution or replacement with a cheaper ingredient can be economically appealing. Food budget for 50% of U.S. families is less than 125 USD per week. A limited budget forces consumers away from balanced meals due to costs or scarcity of the ingredients, towards more high energy dense and unhealthy food types. Government, insurance companies and food companies try to educate consumers through the use of applications to choose for healthy and tasteful food solutions with limited spending. There is an urgent need to make cheap food also healthy.

Replacement or substitution of one or more ingredients or a dish (e.g., a food or drink product) can also offer health benefit. A vast amount of people have a boring single-dimensional food pattern leading to, e.g., excess weight gain. 150 million citizens in the U.S. today are living with some form of a chronic disease or have special dietary restrictions. Since, each of those persons wants to have a personalized solution, there is a need for personalized dietary solutions. 80% of cases of cardiac disease, stroke, type 2 diabetes and 40% of cancers could be avoided through improved lifestyle choices, including those related to diet (see, for example, World Health Organization's 2005 Report on “Preventing Chronic Diseases A Vital investment,” accessible through WHO website as: www<dot>who<dot>int</>chp</>chronic_disease_report</>full_report.pdf<?>ua=1). For example, when a diabetes type 2 patient is diagnosed, a doctor provides caloric and macronutrient recommendations but such recommendations are abstract to the average individual. Therefore, an application is needed for teaching patients to gradually replace certain foods from their diets and replace them with better, healthier choices.

The digital kitchen is becoming a reality. The majority of consumers in the U.S. now use mobile food ordering tools. Mobile users are clearly looking for benefits to motivate them into transitioning their food ordering experience to smart phone applications (see, for example, a 2013 publication at Interactive Advertising Bureau: IAB website at: www<dot>iab<dot>net</>about_the_iab</>recent_press_releases</>press_release_archive</>press_release</>pr-012813_mobile).

Food ingredient substitution in general is known. For example, Taiwan Patent Pub. No. 200926020 and US Pat. Pub. No. 2013/191143 discloses ingredient substitution, but it takes only nutritional and/or preferences into account. U.S. Pat. No. 6,052,667 discloses substituting an ingredient to prevent the aging of another ingredient. US Pat. Pub. No. 2013/0149679 discloses recipe modifications but seems to focus on quantity modification of existing ingredients. None of the publications proposes substitution of one or more ingredient or a dish (e.g., a food or drink product) thereof based on inherent sensory modalities of the ingredient such as taste or flavor. Also none of the publications discloses any method for achieving such substitution.

In sum, there is an urgent need to offer the public knowledge and tools for identifying cheaper and healthier substitute or replacement ingredients for food or drinks, with no or little compromise to the sensory modalities (e.g., taste, flavor, aroma and etc.) of the food.

### SUMMARY

In one aspect, provided herein is a method for identifying a substitute ingredient for a target ingredient. In some embodiments, the method comprises the steps of: comparing one or more sensory parameters representing a sensory modality of the target ingredient with sensory parameters representing the corresponding sensory modality of a plurality of candidate ingredients to calculate a plurality of proximity values, and identifying one or more substitute ingredients among the plurality of candidate ingredients based on a pre-determined cutoff value of proximity value. In such embodiments, each proximity value within the plurality represents a degree of similarity between the sensory modality of the target ingredient and that of a candidate ingredient within the plurality of candidate ingredients;

In some embodiments, the method further comprises a step of converting physicochemical data representing the target ingredient to the one or more sensory parameters representing the sensory modality of the target ingredient.

In some embodiments, the target ingredient has a plurality of sensory modalities and the method further comprises a step of converting physicochemical data representing the target ingredient to one or more sensory parameters representing each sensory modality in the plurality of sensory modalities.

In some embodiments, each sensory modality in the plurality of sensory modalities is further represented by an intensity parameter reflecting the relative strength of the each sensory modality.

In some embodiments, the method further comprises a step of comparing one or more sensory parameters representing each additional sensory modality of the plurality of sensory modalities of the target ingredient with sensory parameters representing the corresponding sensory modality of each candidate substitute ingredient of the plurality of candidate substitute ingredients to calculate proximity values for each the additional sensory modality for each candidate substitute ingredient of the plurality of candidate substitute ingredients; and identifying, for each the sensory modality, one or more substitute ingredients among the plurality of candidate ingredients based on a pre-determined cutoff value of proximity value.

In such embodiments, each proximity value for each the additional sensory modality represents a degree of similarity between each the sensory modality of the target ingredient and that of a candidate substitute ingredient within the plurality of candidate substitute ingredients, and each candidate substitute ingredient has a plurality of proximity values, each corresponding to a sensory modality in the plurality of sensory modalities.

In some embodiments, the method further comprises the steps of combining the proximity values for each candidate substitute ingredient in the plurality of candidate substitute ingredients to calculate a global proximity value for the each candidate substitute concerning all sensory modalities in the plurality of sensory modalities, and identifying one or more global substitute ingredients among the plurality of candidate substitute ingredients based on a pre-determined cutoff value of global proximity value.

In any applicable embodiments, the plurality of sensory modalities comprises any number of sensory modalities. For example, in some embodiments, the plurality of sensory modalities comprises three or more sensory modalities. In some embodiments, the plurality of sensory modalities comprises five or more sensory modalities.

In some embodiments, the combining step further comprises the steps of assigning a parameter to each sensory modality in the plurality of sensory modalities to reflect the relative importance of each sensory modality; adjusting the proximity values for each candidate substitute ingredient in the plurality of candidate substitute ingredients based on the assigned parameter for the corresponding sensory modality; and combining the proximity values, for each candidate substitute ingredient in the plurality of candidate substitute ingredients and in connection with the plurality of sensory modalities, to calculate a global proximity value for the each candidate substitute of the plurality of candidate substitute ingredients.

In some embodiments, the comparing of each sensory modality is based on the relative importance of each sensory modality among the plurality of sensory modalities.

In some embodiments, the one or more sensory parameters used in comparison between the target ingredient and a candidate substitute ingredient are identified based on user preference.

In one aspect, provided herein is a method for identifying one or more substitute dishes for a target dish. In some embodiments, the method comprises the steps of identifying a phase in the target dish, comparing the one or more composite phase sensory parameters for a sensory modality of the plurality of sensory modality of the target dish to those of each of one or more phases in each candidate substitute dish of a plurality of candidate substitute dishes to calculate one or more proximity values for each candidate substitute dish for the sensory modality; combining, for each candidate substitute dish, the one or more proximity values to calculate a global proximity value, thereby rendering a plurality of global proximity values; and identifying one or more substitute dishes among the plurality of candidate substitute dishes based on a pre-determined cutoff value of global proximity value. In such embodiments, the phase has homogenic physicochemical characteristics and sensory parameters and comprises one or more ingredients at respective proportions and prepared according to one or more methods of preparation. Here, the phase is characterized by one or more composite phase sensory parameters corresponding to each of a plurality of sensory modality. Also in such embodiments, each proximity value within the one or more proximity values represents a degree of similarity between the phase in the target dish and each of one or more phases in a candidate substitute dish with respect to the sensory modality.

In some embodiments, the method further comprises the steps of converting physicochemical data representing each ingredient of the one or more ingredients in the phase to one or more sensory parameters representing each ingredient in the one or more ingredients; and combining the one or more sensory parameters representing each ingredient in the one or more ingredients to generate phase sensory parameters for the phase, based on the proportion and the method of preparation of each ingredient in the phase.

In one aspect, provided herein is a method for identifying one or more substitute dishes for a target dish. In some embodiments, the method comprises the steps of identifying a plurality of phases in the target dish, wherein each phase in the plurality of phases has homogenic physicochemical characteristics and comprises one or more ingredients at respective proportions and prepared according to one or more methods of preparation, and wherein each phase is characterized by one or more composite phase sensory parameters; comparing the one or more composite phase sensory parameters for each phase of the plurality of phases of the target dish to those of each of one or more phases in each candidate substitute dish of a plurality of candidate substitute dishes to calculate a proximity value for each phase of each candidate substitute dish, combining, for each candidate substitute dish, the one or more proximity values to calculate a global proximity value, thereby rendering a plurality of global proximity values for the plurality of candidate substitute dishes; and identifying one or more substitute dishes among the plurality of candidate substitute dishes based on a pre-determined cutoff value of global proximity value. In such embodiments, each proximity value represents a degree of similarity between each the phase in the target dish and each of one or more phases in a candidate substitute dish within the plurality of candidate substitute dishes, and each candidate substitute dish has one or more proximity values, each corresponding to a phase in one or more phases in each candidate substitute dish; and

In some embodiments, the method further comprises the steps of converting physicochemical data representing each phase of the plurality of phases in the target dish to one or more phase sensory parameters corresponding to one or more sensory modalities in the each phase; and combining the one or more sensory parameters representing each phase in the plurality of phases to generate one or more composite phase sensory parameters for each phase, based on the proportion and the method of preparation of each ingredient in the each phase.

In some embodiments, the one or more composite phase sensory parameters further comprise an intensity parameter reflecting the strengths of the sensory modalities and a global phase strength of each phase of the plurality of phases.

In any applicable embodiments, the plurality of phases in a target dish can comprise any number of phases. For example, in some embodiments, the plurality of phases in the target dish comprises three or more phases. In some embodiments, the plurality of phases in the target dish comprises five or more phases.

In some embodiments, the one or more composite phase sensory parameters used comparison between a phase in a target dish and one or more phases of a candidate substitute dish are identified based on user preference.

In one aspect, provided herein is a method for identifying a substitute ingredient for a target ingredient. In some embodiments, the method comprises the steps of receiving, from a user via an interface on a computer device, a target ingredient, wherein the target ingredient is entered by the user or selected by the user from one or more ingredients provided at the interface; comparing, at a remote server, one or more sensory parameters of a sensory modality of the target ingredient with sensory parameters of a corresponding sensory modality of each candidate substitute ingredient in a plurality of candidate substitute ingredients to calculate a plurality of proximity values, and determining, at the remote server, one or more substitute ingredients among the plurality of candidate substitute ingredients, based on a pre-determined cutoff value of proximity value. In such embodiments, each proximity value within the plurality represents a degree of similarity between the sensory modality of the target ingredient and that of a candidate substitute ingredient within the plurality of candidate substitute ingredients and the sensory parameters of the plurality of candidate ingredients are stored in a database on the remote server.

In some embodiments, the method further comprises a step of sending, to a user and via the interface, a list of substitute ingredients based on the one or more substitute ingredients.

In some embodiments, substitute ingredients on the list of substitute ingredients are ranked according to their respective proximity values.

In some embodiments, one or more sensory parameters representing a sensory modality of the target ingredient are converted from physicochemical data of the target ingredient.

In some embodiments, the target ingredient has a plurality of sensory modalities and the method further comprises the steps of comparing, at the remote server, one or more sensory parameters representing each additional sensory modality of the plurality of sensory modalities of the target ingredient with sensory parameters representing a corresponding sensory modality of each candidate substitute ingredient in a plurality of candidate substitute ingredients to calculate a proximity value for each comparison; and identifying, at the remote server and for each additional sensory modality, one or more substitute ingredients among the plurality of candidate substitute ingredients based on a pre-determined cutoff value of proximity value. In such embodiments, each proximity value represents a degree of similarity between each additional sensory modality of the target ingredient and a corresponding sensory modality of a candidate substitute ingredient within the plurality of candidate substitute ingredients.

In some embodiments, the method further comprises the steps of calculating, at the remote server and for each candidate substitute ingredient in the plurality of candidate substitute ingredients, a global proximity value based on the proximity values for all sensory modalities associated with the candidate substitute ingredient; and identifying, at the remote server, a list of final substitute ingredients among the plurality of candidate substitute ingredients based on a predetermined global proximity value.

In some embodiments, the method further comprises a step of sending, to a user and via the interface, the list of final substitute ingredients ranked according to their respective proximity values.

In some embodiments, the method further comprises a step of sending, to a user and via the interface, one or more recipes comprising one or more of the substitute ingredients on the list of final substitute ingredients.

In some embodiments, the one or more sensory parameters representing the sensory modalities of the target ingredient or those of each of the one or more substitute ingredients further comprise an intensity parameter reflecting the strengths of the sensory modalities and a global ingredient strength for the target ingredient and each of the one or more substitute ingredients.

In some embodiments, the method further comprises a step of adjusting the proportion of at least one substitution ingredient in the one or more recipe based on the intensity parameters of the target ingredient and of the at least one substitution ingredient.

In any applicable embodiments, the plurality of sensory modalities comprises any number of sensory modalities. For example, in some embodiments, the plurality of sensory modalities comprises three or more sensory modalities. In some embodiments, the plurality of sensory modalities comprises five or more sensory modalities.

In one aspect, provided herein is a method for identifying a substitute dish for a target dish. In some embodiments, the method comprises the steps of receiving, from a user via an interface on a computer device, a target dish, wherein the target dish is entered by the user or selected by the user from one or more dishes provided at the interface, wherein one or more phases are identified in the target dish, wherein each phase comprises one or more ingredients at set proportions and prepared by one or more methods to give rise to homogenic physicochemical characteristics that are converted to one or more composite phase sensory parameters; comparing, at a remote server, one or more composite phase sensory parameters in a phase in the one or more phases of the target dish to those of each of one or more phases in each candidate substitute dish of a plurality of candidate substitute dishes to calculate a proximity value for each comparison, combining, at the remote server and for each candidate substitute dish, the proximity values for the one or more phases thereof to calculate a global proximity value, thereby rendering a plurality of global proximity values for the plurality of candidate substitute dishes; and identifying, at the remote server, one or more substitute dishes based on a pre-determined cutoff value of global proximity value. In such embodiments, each proximity value within one or more first proximity values represents a degree of similarity between the phase in the one or more phases of the target dish and each of one or more phases in a candidate substitute dish. Also in such embodiments, the composite phase sensory parameters of the plurality of candidate substitute dishes are stored in a database on the remote server and;

In some embodiments, the method further comprises a step of sending, to a user and via the interface, a list of substitute dishes based on the one or more first substitute dishes.

In some embodiments, substitute dishes on the list of substitute dishes are ranked according to their respective global proximity values.

In some embodiments, the method further comprises the steps of comparing, at the remote server, each additional phase in the one or more phases of the target dish to each of one or more phases in each candidate substitute dish of the plurality of candidate substitute dishes to calculate a proximity value for each comparison, combining, at the remote server and for each candidate substitute dish, the one or more proximity values for a candidate substitute dish to calculate a global proximity, thereby rendering a plurality of global proximity values; and identifying, at the remote server, one or more substitute dishes based on a pre-determined cutoff value of global proximity value. In such embodiments, each proximity value represents a degree of similarity between each additional phase in the one or more phases of the target dish and each of one or more phases in a candidate substitute dish of the plurality of candidate substitute dishes. In some embodiments, for each candidate substitute dish, there are one or more proximity values.

In some embodiments, the one or more composite phase sensory parameters further comprise an intensity parameter reflecting the strengths of the sensory modalities and a global phase strength of each phase of the plurality of phases.

In some embodiments, the method further comprises a step of sending, to the user and via the interface, a list of final substitute dishes based on the one or more substitute dishes.

In some embodiments, substitute dishes on the list of final substitute dishes are ranked according to their respective global proximity values.

In one aspect, provided herein is a method of presenting sensory data concerning one or more food items. In some embodiments, the method comprises the steps of converting physicochemical data representing a food item of the one or more food items to one or more sensory parameters representing each sensory modality in one or more sensory modalities associated with the food item of the one or more ingredients; and creating a visual representation of sensory parameters for one or more sensory modalities for the food item of the one or more food items.

In some embodiments, the food item is selected from the group consisting of an ingredient, a phase of a dish or recipe, and a dish or recipe. In some embodiments, the visual representation includes indicia corresponding to the relative strength of each of the one or more sensory modalities.

In applicable embodiments, the one or more food items comprise any number of food items. For example, in some embodiments, the one or more food items comprise two or more food items, three or more food items, four or more food items, five or more food items, six or more food items, seven or more food items, eight or more food items, nine or more food items, ten or more food items, or fifteen or more food items.

In some embodiments, the ingredient is included in a phase of a dish or recipe. In some embodiments, the visual representation corresponds to one selected from the group consisting of a phase of the dish, a phase of the recipe, the entire dish, and the entire recipe.

In one aspect, provided herein is a method of generating sensory data. In some embodiments, the method comprises the steps of converting physicochemical data in a knowledge database of ingredients, phases and/or dishes to sensory data; and generating a list of ingredients, phases and/or dishes based on selected characteristics of sensory data.

In one aspect, provided herein is a computer program product for use in conjunction with a computer having a processor and a memory connected to the processor, the computer program product comprising a computer readable storage medium having a computer program mechanism encoded thereon, wherein the computer program mechanism may be loaded into the memory of the computer and cause the computer to carry out the method of any aspect of the invention as disclosed herein.

In any applicable embodiments, the one or more sensory parameters used in comparison between the target ingredient and a candidate substitute ingredient are identified based on user preference.

In any applicable embodiments, the one or more composite phase sensory parameters used comparison between a phase in a target dish and one or more phases of a candidate substitute dish are identified based on user preference.

One of skill in the art would recognize that, when applicable, any embodiments disclosed herein can be used in conjunction with any aspect of the invention.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 illustrates an exemplary embodiment for ingredient and dish substitution.

FIG. 2A illustrates an exemplary ingredient organization, showing how a target ingredient is compared to a candidate ingredient.

FIG. 2B illustrates an exemplary process for ingredient substitution.

FIG. 3A illustrates an exemplary dish organization, showing how a target dish is compared to a candidate ingredient.

FIG. 3B illustrates another exemplary dish organization, showing how a target dish is compared to a candidate ingredient.

FIG. 3C illustrates an exemplary process for dish substitution.

FIG. 4A illustrates an exemplary embodiment for a network system setup.

FIG. 4B illustrates an exemplary embodiment for a network system setup.

FIG. 4C illustrates an exemplary embodiment for a network system setup.

FIG. 5 illustrates an exemplary embodiment for computer network system setup.

FIG. 6A illustrates an exemplary embodiment for visual presentation of sensory parameters.

FIG. 6B illustrates exemplary embodiments for visual presentation of sensory parameters.

### DETAILED DESCRIPTION

Food substitution or replacement disclosed herein offers many advantages, including but not limited to avoiding allergic reactions; decreasing waste of ingredients; reducing overstock; optimizing the cost, profit or margin on a recipe; optimizing the nutritional requirements; maximizing sustainability of a recipe, maximizing or minimizing the quantity of certain ingredients in a recipe, maximizing health benefits of a food or drink recipe, and offering sensorial variation or novelty. Here, “substitute,” “replace” and the variant forms of these words are used interchangeably.

Disclosed herein are the methods and systems for substituting or replacing a food or drink ingredient based on one or more sensory modalities of the ingredient to be replaced. Furthermore, also herein are the methods and systems for substituting or replacing an entire food or drink recipe based on one or more sensory modalities of the food or drink. Also provided here are applications for utilizing such methods and systems. One of skill in the art would understand that any of the embodiments described herein can be used in combination with each other when possible.

As used herein, “sensory modality” refers to a type of physical or physiological phenomenon that one can sense from a food or drink ingredient, for example, by using sensory organs such as the nose, the tongue, the eyes, the skin and etc. Exemplary sensory modalities include but are not limited to a taste, aroma, texture, color, and various combinations thereof. In some embodiments, a flavor is considered the combination of taste, aroma and texture. In some embodiments, an additional parameter, e.g., intensity, is used to reflect the relative strength of the various sensory modalities in connection with the same ingredient.

As used herein, the term “an ingredient” refers to a singular food or drink product, which can exist on its own or is a component of a phase or a component of a dish or recipe. In some embodiments, ingredients are seen as basic elements of a dish (e.g., an entire food or drink product). In some embodiments, the same ingredients, by themselves or after preparation in a dish, exhibit different characteristics; for example, boiling will change the texture and sometimes even taste of an ingredient. As disclosed herein, an “ingredient” is characterized by its macro and micro nutrients. Furthermore, an ingredient can be characterized by a set of physicochemical and sensory parameters. Exemplary sensory parameters include but are not limited to sweetness, sourness, bitterness, saltiness, spiciness, fattiness, protein content, alcohol contents and etc. In some embodiments, sensory parameters are directly converted from physicochemical measurements. For the purpose of this invention, sensory parameters can be grouped by sensory modality. For example, a tomato can be characterized by taste parameters, including but not limited to sweetness, sourness, bitterness and saltiness. Parameters of other modalities (e.g. aroma, texture etc.) can also be quantified.

As used herein, a “dish” refers to a combination of ingredients, which are combined according to a preparation method inherent to the dish. In some embodiments, a “dish” or “system” can be viewed as a collection of one or more homogenic “subsystems” or “phases.” In some embodiments, a “phase” is a combination of ingredients prepared by selected method(s), and all physicochemical and sensorial properties within the reasonable boundaries defining the phase are uniform. Phases are the components of a dish while ingredients are components of a phase. For example, a BLT sandwich is by definition a dish; the bacon, lettuce, tomato, bread and mayonnaise are different phases. Oil, egg yolk and lemon juice are ingredients which are components of the mayonnaise phase. One skilled in the art will notice that lettuce is an ingredient and at the same time a single ingredient phase. In this case, a phase consisting of one ingredient. In other embodiments, a single phase comprises multiple ingredients, such as the mayonnaise phase. Here, “dish,” “recipe,” “food or drink product” or “food or drink preparation” and variants thereof are used interchangeably. In some cases phases are readily available for purchase, in such cases phases can also be called ingredients and thusly treated. For example, mayonnaise is a phase, consisting of oil, egg yolk and lemon juice. Meanwhile mayonnaise can also be viewed as an ingredient since it is readily available for purchase. The term phase is generally reserved to define homogenic subsystems in a dish that are clearly the product of the combination of ingredients and preparation method, e.g. lemon flavored whipped cream.

As disclosed herein, the term “intensity” refers to the measurable amount, presence or perception of a property or characteristic of an ingredient or an entire dish (such as a food or drink). This property or characteristic can be defined at the level of sensory parameters. Sensory parameter intensity can be used to compare sensory parameters of ingredients; for example, via pairwise comparison of calculated distance or proximity metrics. Furthermore, intensity can also be defined on the level of a modality (e.g., taste intensity, aroma intensity, and etc.), an ingredient (e.g., tomato intensity), a phase (e.g. mash potato intensity) and a dish (e.g., BLT sandwich intensity). In some embodiments, modality intensity can be used as a measure for the importance of respective sensory modality in the complete sensory perception of an ingredient or phase.

As used herein, a “target” refers to an ingredient or dish that is to be substituted or replaced; or for which suitable alternatives need to be identified. A “substitute” is an ingredient or dish that is an alternative for the “target.” For examples, when looking for a substitute for thyme (the target), oregano (the substitute) might be a likely candidate.

In one aspect, provided herein are methods and systems for converting physical or chemical data into sensory data. In some embodiments, individual molecular components of an ingredient in a food or drink are identified and/or quantified and categorized into different sensory modalities, including but not limited to, for example, taste, flavor, aroma, texture and etc. In some embodiments, sensory modality data for a target ingredient are further processed to reflect the relative strength or intensity of the modality in the respective ingredient.

In another aspect, provided herein are methods for substituting or replacing a food or drink ingredient or a dish (e.g., a food or drink). In some embodiments, sensory data from a target ingredient or dish is compared with the corresponding sensory data from one or more candidate substitute ingredients or dishes respectively. In some embodiments, pairwise comparison is performed. In some embodiments, multiple pairwise comparisons are used to compare sensory data of multiple sensory modalities.

In another aspect, provided herein are computer-implemented method and system for substituting an ingredient for a food or drink recipe. As disclosed herein, a food or drink ingredient can be substituted by one or more alternative ingredients based on similarities of sensory data between the target and substitute ingredients. In particular, the one or more substitute ingredients have similar sensory parameters with the target ingredient.

In another aspect, provided herein are computer-implemented method and system for providing dishes or recipes with one or multiple substituted ingredients within. In one embodiment a user does not need prior knowledge about the recipe in which the substitute ingredient or multiple substitution ingredients will be used. In another embodiment, such knowledge art can be used to further improve the output of candidate substitute ingredients and finally output the complete dish or recipe with the selected substitute ingredient or ingredients incorporated.

In another aspect, provided herein are computer-implemented method and system for providing sensory information of ingredients, phases and/or dishes as such to interested individuals. The sensory parameters of any ingredient, phase and/or dish can be presented in one form or another to an interested individual. In some embodiments, the presentation forms include but are not limited to a visualization presentation (such as photo, video or animation), a text presentation, an audio presentation, or a combination thereof.

In another aspect, provided herein are computer-implemented method and system for screening a knowledge database of ingredients, phases and/or dishes based on one or more sensory parameters in order to generate a list of ingredients phases and or dishes containing the screened sensory parameters as such. For example: a user is interested in finding ingredients that are sweet, sour and fruity.

In another aspect, provided herein are computer program products that can be used as educational or inquiry tools by interested individuals. The computer program products can be used on any suitable device, including but not limited to a networked device, a local device, a vending machine, a food dispensing machine, a drink dispensing machine, an automated drink maker, an automated cocktail maker, an automated food preparation machine, a desktop computer, a laptop computer, a mobile device, a handheld device, a tablet, an iPad, a Kindle, a cellular phone, a smart phone, a personal digital assistant (PDA), a networked television, a networked media player, and a networked digital video recorder (DVR).

Overall Process for Ingredient Substitution or Dish Substitution

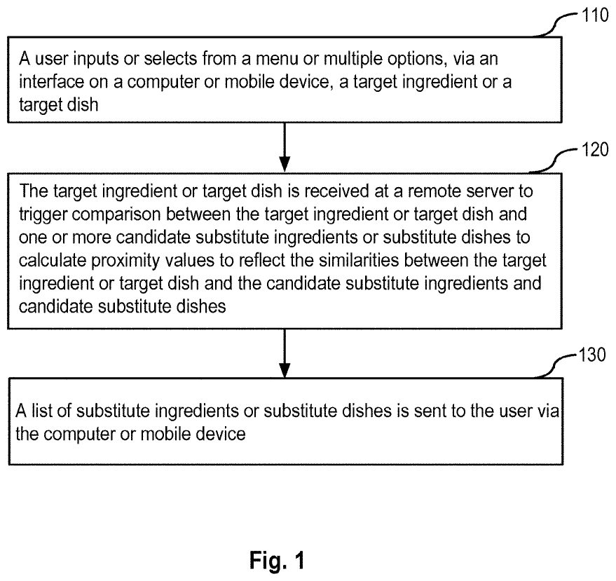

A typical substitution process is outlined in FIG. 1. At one end, a user selects a target ingredient or a target dish as input. The process outputs a ranked list of substitute ingredients or substitute dishes , based on their sensory proximity to the target ingredient or a target dish. The sensory proximity is calculated based on sensory parameters of the both target and substitute ingredients or dishes.

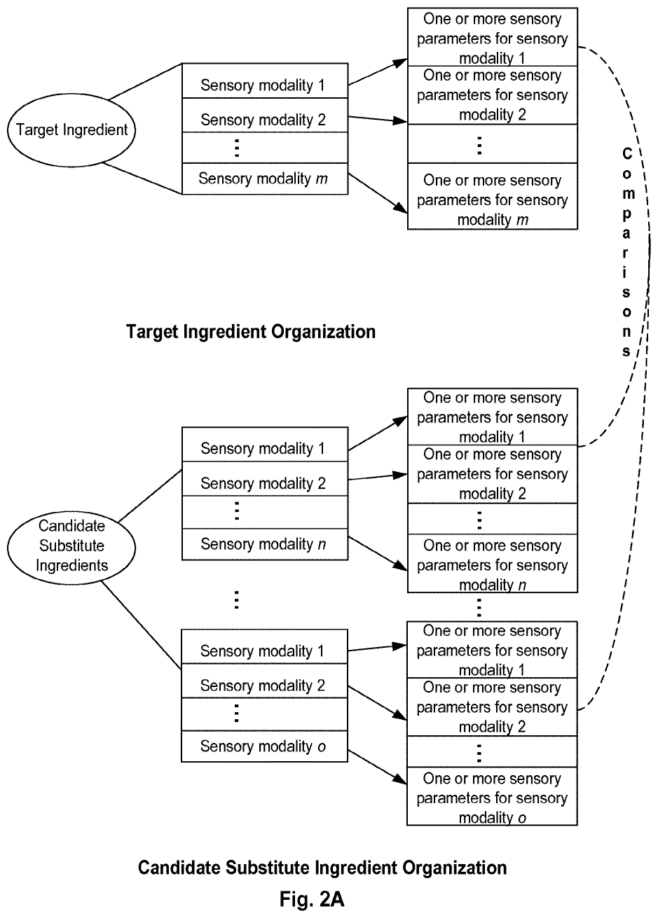

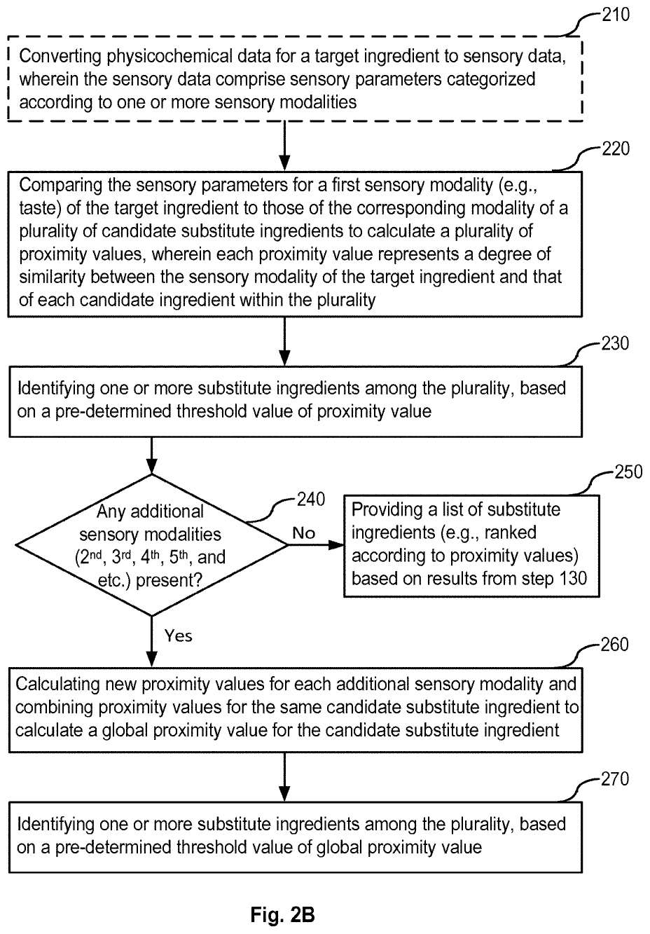

Specifically, ingredient substitution as disclosed herein is based on the sensory modalities and their sensory parameters of a target ingredient, as illustrated by the exemplary embodiments of FIGS. 2A and 2B. Such sensory modalities include but are not limited to the taste, flavor, aroma, intensity, or texture features of the ingredient at issue.

For example, substitution or replacement of individual ingredients is achieved in two steps. The physicochemical data for a target ingredient is first translated into sensory data (e.g., step 210 in FIG. 2B).

After the conversion from physicochemical data to sensory data, sensory data of the target ingredient is compared pairwise to a knowledge database of sensory data of candidate substitution ingredients. For each comparison, a sensory proximity is calculated (e.g., step 220 in FIG. 2B). Candidate substitution ingredients with the highest sensory proximity are identified and presented to the user (e.g., steps 230 and 250 in FIG. 2B). In some embodiments, sensory proximity can be calculated from physicochemical data.

In some embodiments, sensory proximity values are calculated for each modality separately (e.g., step 240 in FIG. 2B). In particular, sensory parameters regarding a specific modality will result in a proximity value for the particular sensory modality (e.g., taste proximity, aroma proximity, texture proximity, and etc.). In some embodiments, the method of calculating modality proximity can differ for each modality.

In some embodiments, sensory modality proximities for multiple sensory modalities can be combined into a global sensory proximity (e.g., step 260 in FIG. 2B). One or more substitute ingredients are then identified based on a pre-determined cutoff value of global proximity value.

In some embodiments, a sensory modality hierarchy can be utilized when comparing the sensory data between different ingredients. When calculating the global sensory proximity, some modality proximities can be weighted more heavily than others. For example, dissimilarities between ingredients in sensory parameters belonging to the taste modality (low taste proximity) can be penalized more severely than dissimilarities in aroma parameters; i.e., dissimilarity in taste can have a more adverse effect on the final proximity value than other modalities, resulting in a bigger increase in the proximity value. In some embodiments, the weight of a modality proximity in the final sensory proximity can be dependent of the relative importance of the respective modality in the target ingredient, which can be assessed by the modality intensities.

In some embodiments, a final sensory proximity, resulting from pairwise comparison of the sensory parameters of target and candidate substitution ingredients, can be used to filter and/or rank a knowledge database of substitution ingredients before outputting a set of substitution ingredients to the user.

In some embodiments, a modality proximity (e.g., taste proximity) rather than final sensory proximity can be used to filter a knowledge database of substitution ingredients before outputting a set of substitution ingredients to the user. Furthermore, a modality proximity (e.g., aroma proximity) rather than final sensory proximity can be used to rank a knowledge database of substitution ingredients before outputting the substitution ingredients to the user.

In some embodiments, a first modality proximity can be used to filter a knowledge database of substitution ingredients and subsequently a second modality proximity can be used to rank a knowledge database of substitution ingredients before outputting the substitution ingredients to the user.

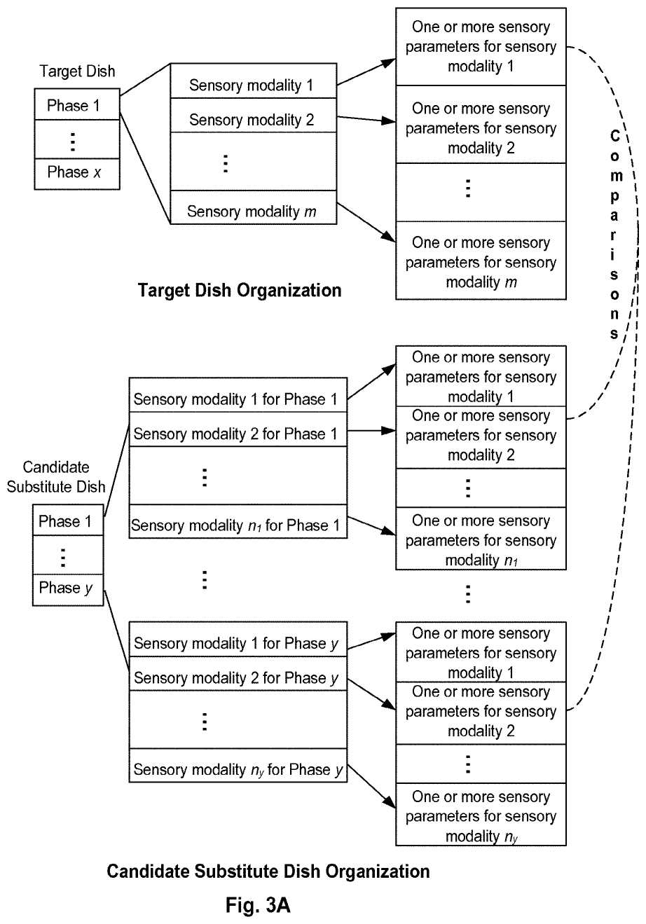

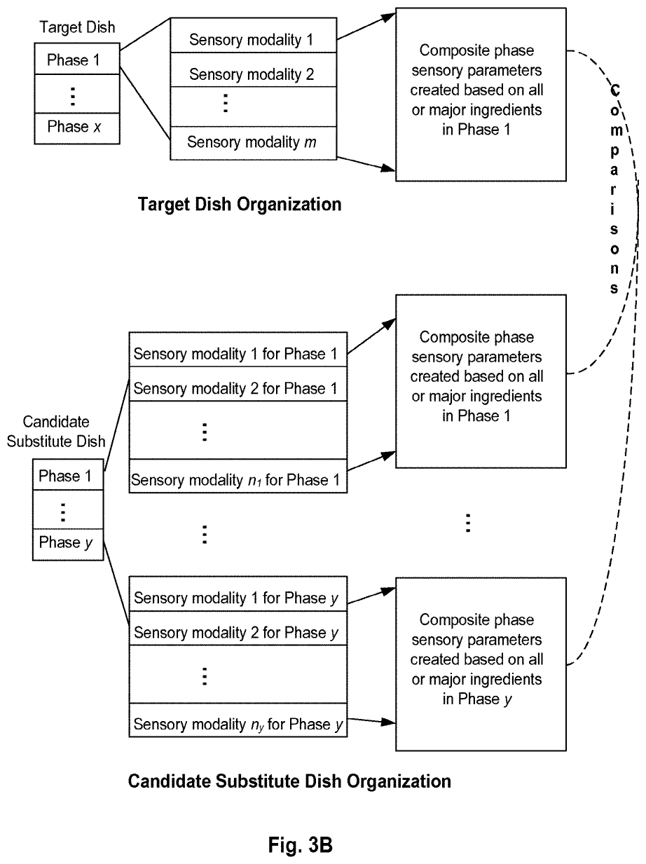

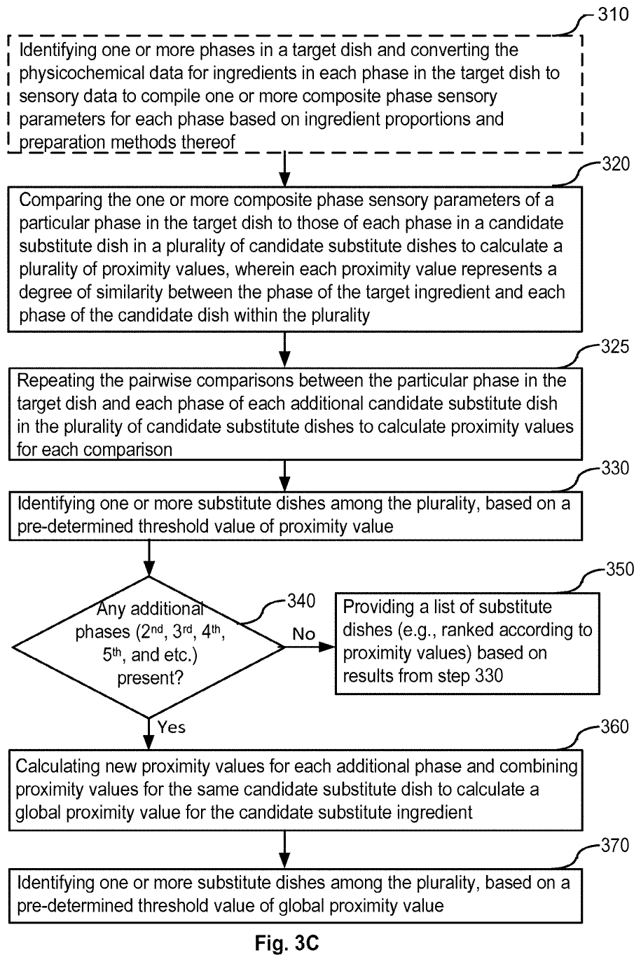

Exemplary embodiments for dish substitution are illustrated in FIGS. 3A and 3B. The overall process shares some similarities to a process for ingredient substitution, but there are also some clear distinctions. In particular, a dish generally includes multiple ingredients prepared according to one or more methods of preparation. As a result, a dish usually includes multiple phases, e.g., a sauce phase, a topping phase, a solid phase and etc.

In some embodiments, each phase includes one or more ingredients that are prepared to become homogenic, having uniform physicochemical characteristics. Consequently, sensory modalities can be defined for a phase instead of each ingredient in the phase (e.g., FIG. 3A). In some embodiments, sensory parameters can be defined for each sensory modality and then compared with the sensory parameters in each phase in a candidate substitute dish to identify the phase that is most similar to the phase in the target dish.

In some embodiments, composite phase sensory parameters can be calculated for an entire phase (e.g., FIG. 3B). In such embodiments, composite phase sensory parameters of a phase in a target dish are compared with the composite phase sensory parameters of each phase in a candidate substitute dish to identify the phase that is most similar to the phase in the target dish.

In some embodiments, the target dish and a candidate substitute dish have different number of phases. In some embodiments, the target dish and a candidate substitute dish have the same number of phases.

In an exemplary process for dish substitution is illustrated in FIG. 3C. At step 310, one or more phases are identified in a target dish and physicochemical data concerning ingredients in each phase are converted to sensory data. In some embodiments, such sensory data comprise one or more composite phase sensory parameters for each phase in the target dish. In some embodiments, composite phase sensory parameters are calculated in two steps. First, physicochemical data of the ingredients in a phase are converted to sensory data at the ingredient level. Secondly, based on the proportions of the ingredients involved and respective preparations methods thereof, ingredient sensory parameters are combined to generate composite phase sensory parameters. By definition, a phase is homogenic and thus has uniform phase sensory parameters. As such, it is possible to characterize a phase using composite phase sensory parameters.

At step 320, composite phase sensory parameters for a particular phase in the target dish are compared to the composite phase sensory parameters of each phase in a candidate substitute dish. A proximity value is calculated for each pairwise comparison. Based on the proximity values, the phase in the candidate substitute dish that is most similar to the particular phase in the target dish is identified.

At step 325, pairwise comparisons are carried out between the particular phase of the target dish and each phase in all other candidate substitute dishes. For each comparison, a proximity value is compared to reflect the similarity between the two phases being compared.

At step 330, one or more substitute dishes are provided based on a pre-determined cutoff value of proximity value. At this point, only one phase is compared; so it is likely that the substitute dishes do not truly resemble the target dish. In some embodiments, however, when a dish is dominated by one dish (e.g., mash potatoes or a puree soup), a single phase comparison scheme is sufficient for identifying substitute dishes (e.g., step 350).

At step 340, with more complex dishes, it is often necessary to carry out multiple phase comparisons. Basically, steps 320 through 330 are carried out for every phase in a target dish against every phase of every available candidate substitute dish.

At step 360, for the same candidate substitute dish, proximity values for all its phases are combined to calculate a global proximity value to reflect the overall similarity between the target dish and the candidate substitute dish. The same is carried out to calculate a global proximity value for each available candidate substitute dish.

At step 370, one or more substitute dishes are identified if their corresponding global proximity value is equal or greater than a pre-determined cutoff value of global proximity value.

### Converting Physicochemical Data to Sensory Data

Physicochemical Data and Sensory Data

Physicochemical data include the quantifiable measurements of the molecular components of an ingredient or ingredients in a dish (e.g., a food or drink). The following table illustrates some exemplary physicochemical data and the sensory data to which these physicochemical data correspond.

TABLE 1
Exemplary physicochemical data of components in ingredients
and exemplary corresponding sensory parameters.

| Physicochemical data (per 100 g of product) | Sensory parameters |
|---|---|
| Grams of NaCl equivalent x 10 | Saltiness |
| Grams of Citric acid equivalent x 10 | Sourness |
| Grams of Sucrose equivalents | Sweetness |
| Grams of total fat content | Fattiness |
| Grams of capsaicin or isothiocyanate | Pungency |
| Grams of MSG equivalents x10 | Umami |
| Grams of Ethanol | Alcohol content |
| Grams of menthol equivalents (isomers of menthol | Cooling effect |
| and other derivatives, camphor, 1,8-cineol, . . . | |
| or grams of fat at melting temperature body) | |
| Grams of total protein content | Protein content |

In some embodiments, physicochemical data is translated into sensory parameters and their corresponding sensory parameters intensities; e.g. sucrose amongst others corresponds to sweetness and benzaldehyde amongst others corresponds to a combination of aroma parameters such as fruity, floral and etc. (see Table 3).

In some embodiments, interactions between sensory parameters; e.g., the effects sweetness has on spiciness and vice versa can be taken into account during the translation from physicochemical to sensory data. For example, the relative presence of sweetness and spiciness can affect the intensity of each parameter.

In some embodiments, each sensory parameter is a combination of several physicochemical characteristics. For example, sweetness can also come from fructose, lactose, and etc., in addition to sucrose. Fruity flavor can also come from ethyl butanoate, isopentyl acetate, and etc. An ingredient always contains a range of different taste and aroma molecules. In some embodiments, some physicochemical characteristics relate to several sensory parameters.

In some embodiments, physicochemical data are converted into sensory data with a simple linear transformation. In some embodiments, physicochemical data are converted into sensory data with a logarithmic transformation. In some embodiments, physicochemical data are converted into sensory data with a sigmoidal transformation. In some embodiments, multiple types of transformation are applied to optimize conversion of physicochemical data to sensory data.

In some embodiments, physicochemical data are used directly without being converted into sensory data, in the process for ingredient or dish substitution.

In preferred embodiments, the process for ingredient substitution requires that the sensory parameters are standardized by “humanly determined” intensity; i.e., when an intensity score is assigned to a particular taste parameter, it should represent the same level of intensity perceived for a different taste parameter. For example, an acidity intensity score of 50 should have a real life intensity that is comparable to the real life intensity of a saltiness intensity score of 50. Such standardization can be accomplished during the conversion from physicochemical data to sensory data.

Alternative methods for converting physicochemical data to sensory data can also be used. For example, sensory data can be directly measured by a trained expert panel. In such embodiments, the process for ingredient substitution can be modified.

Aroma Modality

In some embodiments, aroma parameters also known as aroma descriptors are used to describe a particular aspect of the aroma modality of an ingredient. In some embodiments, aroma descriptors are sourced from scientific literature or publication. In some embodiments, aroma descriptors are sourced through actual scientific research including gas chromatography organoleptic (GC-O) analysis. For example, the same aroma molecule can be described using different descriptors (see Table 2).

TABLE 2
Exemplary aroma parameters for an
exemplary molecular component.

| | | Concentration |
|---|---|---|
| Aroma molecule | Aroma parameter | (mg/kg) |
| benzaldehyde | floral | 4 |
| benzaldehyde | herbal | 4 |
| benzaldehyde | warm | 4 |
| benzaldehyde | fruity | 4 |

In some embodiments, physicochemical data such as data of aroma molecules and respective concentrations (e.g., in standardized units mg/kg) are available for an ingredient. In some embodiments, an ingredient in a food or drink have multiple aroma molecules, as illustrated in the following table.

TABLE 3
Exemplary chemical data.

| Food or drink | | Concentration |
|---|---|---|
| ingredient | Aroma molecule | (mg/kg) |
| Almond | benzaldehyde | 4 |
| Almond | α-ionone | 0.5 |
| Almond | β-ionone | 0.5 |
| Almond | 2-pyrrolecarbaldehyde | 8 |
| Almond | 2-acetylpyrrole | 0.5 |
| Almond | (2-furyl)pyrazine | 1.5 |
| Almond | 2-(2-furyl)-3- | 2 |
| | methylpyrazine | |
| Almond | trimethylpyrazine | 30 |
| Almond | 6,7-dihydro-5-methyl-5H- | 0.001 |
| | cyclopentapyrazine | |
| Almond | furfural | 9 |
| Almond | 5-(hydroxymethyl)furfural | 9 |
| Almond | furfuryl alcohol | 16 |
| Almond | methyl 2-furancarboxylate | 1.5 |
| Almond | furfuryl acetate | 3 |

In some embodiments, aroma molecule data of an ingredient can be converted into aroma parameter data. In an exemplary embodiment, a given aroma molecule is assigned without calculations to its corresponding aroma parameters and the original concentration of the aroma molecule is used to reflect the absolute strength of the corresponding aroma parameters.

In some embodiments, an aroma parameter can be assigned to multiple aroma molecules of a food or drink product. In such a case, concentrations of the corresponding aroma molecules can be added together to reflect the combined effect of these aroma molecules on the aroma parameter. This is done to create an intuitive parameter to provide a quantitative representation of the strength or extent of presence of a descriptor (e.g., nuttiness) in a food or drink. Likewise, in some embodiments, an aroma molecule might be assigned to multiple aroma parameters.

In some embodiments, absolute strength of each aroma parameter in an ingredient is adjusted as relative strengths in accordance with their actual presence in the ingredient. According to this method, all these relative strengths add up to 100%. In the following table, almond is characterized with different aroma parameters. In some embodiments, aroma parameters are named after other ingredients (e.g., peanut, coffee, bread, cocoa and etc.). Persons practiced in the art will recognize this as a standard procedure.

TABLE 4
Exemplary ingredient aroma parameters.

| Food | aroma paramerter | relative strength |
|---|---|---|
| Almond | Musty | 63.5 |
| Almond | Nutty | 34.501 |
| Almond | Powdery | 34.5 |
| Almond | caramellic | 34 |
| Almond | Sweet | 33.001 |
| Almond | Peanut-like | 30.001 |
| Almond | Roasted | 30.001 |
| Almond | Earthy | 30.001 |
| Almond | potato-like | 30 |
| Almond | cocoa-like | 30 |
| Almond | Bready | 25.5 |
| Almond | Brown | 25 |
| Almond | coffee-like | 24.001 |
| Almond | Alcoholic | 16 |
| Almond | sulfureous | 16 |
| Almond | Chemical | 16 |
| Almond | estery | 16 |
| Almond | woody | 10 |
| Almond | phenolic nuance | 9 |
| Almond | baked bread | 9 |
| Almond | waxy | 9 |
| Almond | fatty | 9 |
| Almond | fragrant | 9 |
| Almond | beefy | 8 |
| Almond | fruity | 8 |
| Almond | benzaldehyde | 4 |
| Almond | cherry-like | 4 |
| Almond | banana-like | 3 |
| Almond | horseradish-like | 3 |
| Almond | orris-like | 1 |
| Almond | floral | 1 |
| Almond | walnut-like | 0.5 |
| Almond | tropical | 0.5 |
| Almond | floral | 0.5 |
| Almond | seedy | 0.5 |
| Almond | violet-like | 0.5 |
| Almond | berry-like | 0.5 |
| Almond | dry | 0.5 |
| Almond | licorice-like | 0.5 |
| Almond | coumarin | 0.5 |
| Almond | corn-like | 0.001 |
| Almond | savory | 0.001 |
| Almond | baked potato-like | 0.001 |
| Almond | grain | 0.001 |
| Almond | toasted | 0.001 |
| Almond | meaty | 0.001 |

Texture Modality

In some embodiments, texture parameters (also known as texture descriptors) are used to describe a particular aspect of the texture modality of an ingredient. In some embodiments, texture parameters are sourced from scientific literature or publication.

In some embodiments, physicochemical data concerning texture can be obtained through instrumental analysis and converted to sensory texture data. Alternatively, sensory texture data can be directly measured by a trained expert panel. Some pure physicochemical parameters can be included in the texture modality such as serving temperature, state of aggregation or colloidal parameters. The following is an exemplary list of sensory texture parameters, supplemented with physicochemical parameters:

TABLE 5
Exemplary ingredient texture parameters.
Texture parameters

Aggregation state
Colloidal state
Water content
Hardness
Brittleness
Chewiness
Flexibility
Viscosity
Adhesiveness
Airiness
Fattiness
Astringency
Mouthcoating
Temperature
Aggregation state
Colloidal state
Water content
Hardness

Intensities

As disclosed herein, intensities of modalities can be calculated utilizing modality parameter intensities. In some embodiments, modality intensity is calculated utilizing physicochemical data directly. For example, the flavor intensity of an ingredient can be calculated using aroma concentration data of the ingredient. In another embodiment, modality intensities can be directly measured by a trained expert panel.

In some embodiments, the concentration or relative quantity of an ingredient in a recipe reflects a parameter intensity, a modality intensity or a global intensity of the ingredient. For example, the modality can be a flavor, aroma, taste, texture, and etc. In such embodiments, a parameter intensity, modality intensity or global intensity can be quantified utilizing the relative quantity of the respective ingredient in a recipe.

In some embodiments, more complex algorithms are used to reflect the relative strength of a modality associated with an ingredient in a dish containing multiple ingredients. For example, concentrations or relative quantities of ingredients or individual molecular components are converted to Odor Activity Values (OAV), which are then converted to aroma intensity values. In some embodiments, individual intensity values are further converted to a global intensity value. In some embodiments, this approach is particularly useful for assessing modalities such as aroma and flavor.

Converting raw input data to OAV values: In some embodiments, quantitative data of aroma molecule of an ingredient are converted to OAV values. For example, ingredients and their molecular components and concentrations, are combined with a “threshold of detection” data to create OAV values to reflect the relative strength of the molecular component in an ingredient.

Finding the relevant threshold values: In some embodiments, relevant threshold values for specific molecules are determined in different contexts, for example, using a context matrix such as water, beer, dark ale, fat, and etc. This is because different molecules have different extraction efficiencies in different media. For example, one can detect “molecule X” at lower concentrations when in water than when in fat. In some embodiments, for each molecule in an ingredient, an algorithm searches for available threshold data of a molecule for the relevant matrix as deep as the data goes. Alternative media are searched in a sequential manner. For example, for “molecule X” in an ingredient that features a dark ale, threshold data for “molecule X in dark ale” is first searched. If none is found, threshold data for “molecule X in ale” is searched. As long as insufficient threshold data is found, the algorithm repeats its search with a more generalized matrix. This ensures that the most relevant threshold is used for further calculations out of all available threshold data. As disclosed herein, threshold data are initially determined through empirical research. In some embodiments, threshold values relate to the perception limit of a human to one or more ingredients or the molecular components therein. Threshold values are set to exclude insignificant molecular components from consideration and hence focus the sensory parameters on molecular components that truly contribute to human perception of the ingredients (or dishes). In some embodiments, the threshold values have been determined in existing physicochemical data that are available in public accessible databases. In some embodiments, publically available data are re-organized, sometimes modified, and compiled into new threshold values and stored in dedicated database on a remote data server.

Building an overall threshold: In some embodiments, different threshold measurements gathered through one or more previous algorithms are combined using various mathematical techniques (e.g., mean, median, geometric mean, maximum measurement, minimum measurement) to produce an overall threshold value for “molecule X in an ingredient Y.” In some embodiments, overall threshold values are determined for each molecular component in an ingredient Y.

Creating an OAV value: In some embodiments, for a particular molecule in an ingredient, its concentration is divided by its corresponding overall threshold value to create a starting OAV value. This value can be capped off at a maximum, set to zero if the value falls below a minimum, or used as an input into an exponential/logarithmic function to skew values into workable OAV values.

Converting OAV values to an intensity value: In some embodiments, a starting intensity is obtained by applying a power law algorithm, e.g., Stephen's Power Law (SPL), to the OAV values. The power law exponent is dependent on chemical parameters including the number of carbon atoms and the chemical group of the molecule in question. Because the SPL is designed to work under a smaller range of OAV differences than present in our foods, the starting intensity obtained via SPL is subject to further processing. In some embodiments, further processing takes place through means of a skewed sigmoidal or logarithmic fit to the power law to stimulate a more reasonable high-end values, or even a maximum value, representing saturation of the human olfactory system. In some embodiments, methods with hard maximums (“cut-offs”) or logarithmic values of the starting intensity are used for further processing as well. These functions are applied to the starting intensity, in order to obtain one final intensity per molecule concentration in a particular ingredient. All of the above functions include constants that can be fine-tuned by fitting these functions to experimental testing data. In some embodiments, the experiments include user perception evaluations where participants are asked to rate the intensity of a molecule at various concentrations in a particular matrix.

Converting a molecule's intensity in an ingredient to aroma intensity: In some embodiments, intensities of individual molecules are combined through Euclidean distance, Manhattan distance or fractional distance metrics, using the maximum individual intensity, or summing up all of the individual intensities. In some embodiments, a different approach, taking an arithmetic or geometric mean of all individual intensities which are above a certain minimum value, has also been used.

In some embodiments, another approach is applied by combining OAV values determined with the use of molecule's aroma parameters. In such embodiments, the first step is once again obtaining the OAV values through concentration and threshold data of molecules in an ingredient, using, for example, method disclosed herein. Each molecule can be described with a series of aroma parameters and their respective contributions (e.g., described as weight values) in the aroma of the molecule. For example, molecule X can be considered “cheesy with a weight value w1, spicy with a weight value w2, and roasted with a weight value w3, and etc.,” where the respective weight values add up to one. In some embodiments, the weight values are stored in an aroma parameter vector (w1, w2, w3 . . . ) per molecule, independent of the ingredient in which the molecule is found.

In some embodiments, for each molecule in an ingredient, the molecule's aroma parameter vector is multiplied by the OAV value to create an OAV vector in the aroma parameter space of the molecule in an ingredient.

In some embodiments, aroma parameter intensities of a molecule in an ingredient are once again calculated through a power law or using methods described herein, and stored as the aroma parameter intensity vector for the particular molecule in an ingredient.

In some embodiments, the aroma parameter intensity of an ingredient on a certain aroma parameter level can be calculated by combining the aroma parameter intensities of molecules in the ingredient, using the various techniques described herein; for example, as geometric or arithmetic means of intensities above a certain level, or as fractional distance metrics. The ingredient now has a total aroma parameter intensity vector.

Using combination techniques disclosed herein or known in the art, this vector is used to calculate aroma intensity of an ingredient.

According to the method and system disclosed herein, intensity is used in conjunction with other sensory modalities. For example, a taste intensity can be calculated by combining all taste parameter intensities.

In some embodiments, a global intensity can be calculated by combining all modality intensities of a particular ingredient. Furthermore, a phase intensity can be calculated by combining all participating ingredients, their quantities in the phase and their preparation methods. In some embodiments, a dish intensity can be calculated utilizing all participating phases and their respective quantities.

In further embodiments, the ingredient intensity of a substitution ingredient can be utilized to give an indication of the proportion of the substitution ingredient that needs to be maintained to correctly substitute a target ingredient.

### Ingredient Substitution Based on Sensory Data

After the relevant physicochemical data are converted to sensory data (e.g., sensory parameters), comparisons are carried out between a target ingredient and substitute ingredient. In some embodiments, sensory data (e.g., sensory parameters) representing one sensory modality are used in the comparison. In some embodiments, a sensory modality encompass a multitude of sensory data; including but not limited to data relating to, for example, two or more sensory parameters, three or more sensory parameters, four or more sensory parameters, five or more sensory parameters, six or more sensory parameters, seven or more sensory parameters, eight or more sensory parameters, nine or more sensory parameters, ten or more sensory parameters, and etc. When multiple sensory parameters fall within the same sensory modality, pairwise comparisons between a target ingredient and a candidate substitute ingredient are carried out with respect to each sensory parameter.

In some cases, not all available modalities are needed for adequate ingredient substitution. In some embodiments, sensory data (e.g., sensory parameters) representing two or more sensory modalities are used in the comparison. In some embodiments, the sensory data (e.g., sensory parameters) of sensory modalities include data for three or more modalities, four or more sensory modalities, five or more sensory modalities, six or more sensory modalities, seven or more sensory modalities, eight or more sensory modalities, ten or more sensory modalities, and etc. When two or more sensory modalities are involved, pairwise comparison can take place in a sequential manner; for example, according to a hierarchical scheme or in a random order.

In order to evaluate the differences between a target ingredient and a candidate substitute ingredient, in some embodiments, the dissimilarities between the sensory parameters for the target ingredient and the candidate substitute ingredient are represented by a sensory proximity value or a sensory distance value. Sensory proximity can be calculated for each modality separately; e.g., only regarding sensory parameters belonging to a specific modality will be used to give rise to a sensory proximity for the particular modality (e.g., taste proximity, aroma proximity, texture proximity etc.). Sensory modality proximities can be combined into a global sensory proximity. The magnitude of the global sensory proximity is proportional to degree of similarity between sensory parameters of the compared ingredients. The candidate substitute ingredient having the highest proximity will be ranked the best substitution ingredient. For the purpose of clarity, “proximity” is the reverse measure of “distance.” As such, when a distance value is calculated, ingredients having the smallest distance will be the most similar.

Various methods of comparison can be used for calculating a proximity measure. The method of calculating modality proximity can differ for each modality. Exemplary methods that are used include but are not limited to: Euclidean distance, Manhattan distance, discrete distance or fractional distance metrics and etc.

In some embodiments, a clustering mechanism is used to compare corresponding sensory modality data between a target ingredient and each one of possible candidate substitute ingredients. For example, two-dimensional map or a map with three or more dimensions can be used to illustrate how closely ingredients or molecular components of an ingredient are matched.

Exemplary Taste Proximity

In some embodiments, the taste proximity is used. In such embodiments, physicochemical data concerning the taste of one or more ingredients are converted to sensory data (e.g., taste parameters).

In some embodiments, physicochemical data are always defined by content; for example, the amount of a particular ingredient or equivalent thereof per 100 g of product. In some embodiments, physicochemical data can be extracted from known nutritional data. In some embodiments, physicochemical data can be analyzed using standard analytical techniques or tools. In some embodiments, physicochemical data can be obtained through new techniques like near infrared combined with powerful algorithms in devices like Scio.

As disclosed herein, taste parameters of any ingredient can be seen as dimensions in an n-dimensional Euclidian taste space. Metric functions such as but not limited to Euclidean distance, Manhattan distance, discrete distance or fractional distance metrics can be used to calculate taste proximity between target and substitution ingredients.

Exemplary Aroma and Texture Proximity

In one embodiment, aroma proximity and/or texture proximity can be calculated in a similar fashion as taste proximity. In such embodiments, physicochemical data concerning the aroma and/or texture of one or more ingredients are converted to sensory data.

According to the exemplary aroma and texture proximity disclosed herein, aroma parameters and/or texture parameters of any ingredient can be seen as dimensions in an n-dimensional Euclidian space. Metric functions such as but not limited to Euclidean distance, Manhattan distance, discrete distance or fractional distance metrics can be used to calculate aroma and or texture proximity between target and substitution ingredients.

### Exemplary Global Sensory Proximity

In some embodiments, when multiple sensory modalities are used to identify one or more substitute ingredients for a target ingredient, one or more modality proximities are constructed to facilitate the identification of proximate substitute ingredients.

In some embodiments, two or more sensory modalities are used to find suitable substitution ingredients. In some embodiments, three or more sensory modalities are used to find suitable substitution ingredients. In some embodiments, four or more sensory modalities are used to find suitable substitution ingredients. In some embodiments, five or more sensory modalities are used to find suitable substitution ingredients. In some embodiments, auxiliary parameters (e.g., intensity values) in connection with one or more sensory modalities are also incorporated in process for substitution.

In some embodiments, a global sensory proximity can be calculated by combining the calculated modality proximities. In this combination, the modality proximities can be weighted according to the importance of the respective modalities in the target ingredient. Relative modality intensity can be a measure of modality importance.

### Exemplary Ingredient Substitution Algorithm

In an exemplary and non-limiting embodiment, a target ingredient is selected by a user. In a first step, a list of candidate substitution ingredients is generated based taste proximity towards the target ingredient. In some embodiments, the number of candidate substitution ingredients in the list from the first step can be adjusted by changing the threshold taste proximity values. For example, a threshold value can be set so that only the top 100 or fewer, 80 or fewer, 60 or fewer, 50 or fewer, 40 or fewer, 30 or fewer, 20 or fewer, or 10 or fewer candidate substitution ingredients are withheld. In some embodiments, a user can select from a menu the number of top ranked candidate substitution ingredients or dishes that he or she would like to receive.

In a second step the candidate substitution ingredients from step 1 are ranked according to their global proximity value towards the target ingredient. The resulting ranked list is outputted to a user so the user can select one or a multitude of substitution of ingredients.

In some embodiments, a particular hierarchical strategy can be implemented based on the assumption that a suitable substitution ingredient should primarily be similar in terms of taste, and only secondly in terms of aroma and etc. Therefore, the substitution ingredients are initially filtered based on taste proximity alone and in a second stage aroma or other modality is added. The strategy that taste takes priority over aroma is supported by many examples; for example, when lemon is defined as the target ingredient, it should be substituted by an ingredient with similar sourness in the first place. Other features such as aroma are of secondary importance. Hence, a high aroma proximity cannot compensate for a low taste proximity. This strategy is in most common cases necessary because taste integrity of the resulting dish with the substituted ingredient is subconsciously desirable. For example; a whisky sour with lime juice instead of lemon juice will have a taste profile that is similar to the original dish and will be appreciated, even if the aroma profile is different from the original. In a different example, when lemon syrup is selected as substitution for lemon juice, taste integrity is not respected in the substitution. In such a case, the recipe's taste balance can be detrimentally altered, the new dish is consequently unlikely to be accepted by an average consumer.

In another exemplary and non-limiting embodiment, the texture modality is added in the ingredient substitution algorithm in addition to taste and aroma. In some cases, it is desirable to have a strong textural proximity towards the target ingredient in those cases where textural integrity of the final dish with the substituted ingredient is highly desirable.

In a more specific embodiment, this algorithm can be altered, especially in cases where taste similarity and taste integrity is not a primary importance, e.g., when replacing blueberries by chocolate chips in a muffin.

In one embodiment, participation of modalities in the substitution algorithm is dependent of the modality intensities of the target ingredient. For example, when searching for a substitution ingredient for potato chips, texture modality will be added to the algorithm because the texture intensity is relatively high in potato chips. When searching for a substitution ingredient for lemon juice, taste modality will be taken into account and texture will be omitted since the taste intensity is relatively high in lemon juice and the texture intensity is relatively low.

In a more specific embodiment, a user can choose to substitute an ingredient without prior knowledge of the final dish or recipe in which the ingredient will be used. Alternatively, in another specific embodiment, the user can choose to substitute an ingredient with prior knowledge of the final dish or recipe in which ingredient will be used. In some embodiments, such prior knowledge can be utilized to further improve the substitution algorithm and thus its output. In such a case, the final output can be the recipe with the user selected substitution ingredient or multiple substitution ingredients.

In one embodiment, the resulting list of suitable substitution ingredients for a target ingredient can be used to screen a knowledge database of recipes for recipes primarily containing the target ingredient, secondarily containing any of the suitable substitution ingredient. For example, when searching a database of recipes for cocktails with lemon juice, one might primarily want to receive a list of cocktails containing lemon juice, but there can also be an interest in cocktails containing suitable alternatives of lemon juice, for example cocktails containing lime juice. This embodiment can become increasingly interesting when applying extended ingredient search queries on a knowledge database of recipes. Such an embodiment can also be combined with the process for dish substitution.

### Overall Process for Dish Substitution

In one embodiment, one might be interested in finding complete dish substitutes rather than individual ingredient substitutes. The process for dish substitution is an extension of the process for ingredient substitution. A “dish” refers to a combination of ingredients, which are combined according to a preparation method inherent to the dish. As disclosed herein, a “dish” or “system” can be viewed as a collection of one or more homogenic “subsystems” or “phases.” As used herein, a “phase” is a combination of ingredients and preparation methods and all physicochemical and sensorial properties within the reasonable boundaries defining the phase are uniform.

In most cases, it is impossible to define a single set of sensory parameters for a given dish since in most cases a dish is a heterogenic system. Therefore any recipe should be viewed as a collection of homogenic subsystems, e.g., phases, each of which can be described by a set of sensory attributes.

In some embodiments and under the assumption of summability, the sensory parameters of a phase can be calculated based on sensory perception; e.g., the sensory parameters of its participating ingredients and their respective proportions in the phase. Furthermore, under the assumption that the sensory perception of an ingredient in a phase is proportional with its relative share in that phase. Furthermore, preparation method can be taking into account when calculating the sensory perception of a phase, preparation method can be seen as a final transformation of the sensory parameters of the phase. The previous is only true for taste and aroma modality.

The texture modality does not follow the assumption of summability. The texture parameters of a phase are generally the result of preparation method and textural properties of the participating ingredients in that phase, interactions are extremely complicated, simple summability of parameters of participators and preparation method would be an oversimplification. Therefore the textural modality in a dish is always defined on the level of phases and not on the level of the participating ingredients, exception is of course when dealing with single ingredient phases, for example melon balls.

In some embodiments, food phases are characterized in the same sensory parameters as ingredients. A food phase can be regarded as a single unit in the process for dish substitution. But for finding alternative dish suggestions, all relevant phases of the target dish must be taken into account as well as all the phases of the substitution dish. The ideal alternative dish would be a dish were all its phases are pairwise ideal substitutions of the phases of the target dish.

The objective of the process for dish substitution is to find similar dishes that are similar to a target dish. In some embodiments, the general strategy is first to define homogenic phases in the target and substitution dishes, which are characterized by homogenic aroma, taste and texture. In a next stage, the general process is to apply ingredient substitution to each phase of the dish, where each phase comprising one or more ingredients. Thirdly, proximity scores from all comparison are combined.

In order to achieve this, all phases of the target dish must be compared to all the phases of the substitution dish. In some embodiments, a first phase in the target dish is compared individually with each of all phases in a candidate substitute dish to identify the optimally match phase. This process is then carried out for all phases in the target dish. The global sensory proximities for each input phase towards all phases of the substitution dish are combined to obtain a global sensory proximity for the substitution dish.

In another embodiment, the process for ingredient substation can also be applied for finding a suitable phase alternative for a target phase, with or without prior knowledge about the recipe of dish the phase is present in.

### Special Case: Cocktail Substitution

Cocktails are within reasonable boundaries homogenic systems usually containing only a single phase, when not taking into account any garnishes, foams, ice phases of layered cocktails. Cocktails can be regarded as single phase dishes where the texture modality is of relative lesser importance. This reduces the process for dish substitution to a process that is very similar to ingredient substitution, since only 1 phase needs to be regarded in the taste and aroma modality.

In some embodiments, a target cocktail is regarded as a single unit in the process for ingredient substitution, which is then utilized to obtain a list of alternative cocktail recommendations. For that purpose, the process for ingredient substitution can be run on cocktail data instead of ingredient data. A similar approach can be utilized to find suitable phase alternative for a target phase.

### Computer Implementations

Further provided herein are computer methods and systems for implementing the methods disclosed herein.

In some cases, computer-based system and devices provide backend functions for implementing the methods disclosed herein. Exemplary functionalities include but are not limited to receiving data input, inquiries and request from a user; storing data and information; generating data and information by searching or screening existing database; carrying out analysis; creating and maintaining user profiles; and providing and presenting information to users either through system automation or in response to user inquiries. In some embodiments, many or all of such functionalities are carried out on a remote server.

In other cases, computer-based system and devices provide user access to stored information or analytical results, via, e.g., a user interface, either as a dedicated computer program product (e.g., a mobile app) or as an interface on a network platform (e.g., a web interface).

For example, the ingredient substitution and dish substitution methods as disclosed herein can be implemented by a dedicated computer program such as a mobile app. Alternatively, a user can search for substitute ingredient or substitute dish on a web interface.

Provided herein are computer-implemented methods and systems for providing dishes or recipes with one or multiple substituted ingredients within. In some embodiment, a user does not need prior knowledge about the recipe in which the substitute ingredient or multiple substitution ingredients will be used. For example, a user has an allergy to a certain ingredient and can inquire alternatives or substitutes to the particular ingredient. In some embodiments, one or more recipes for food or drink are provided at the same time when the alternative or substitute ingredients are suggested.

In some embodiments, such prior knowledge can be used to further improve the output of candidate substitute ingredients and finally output complete dishes or recipes with the selected substitute ingredient or ingredients incorporated.

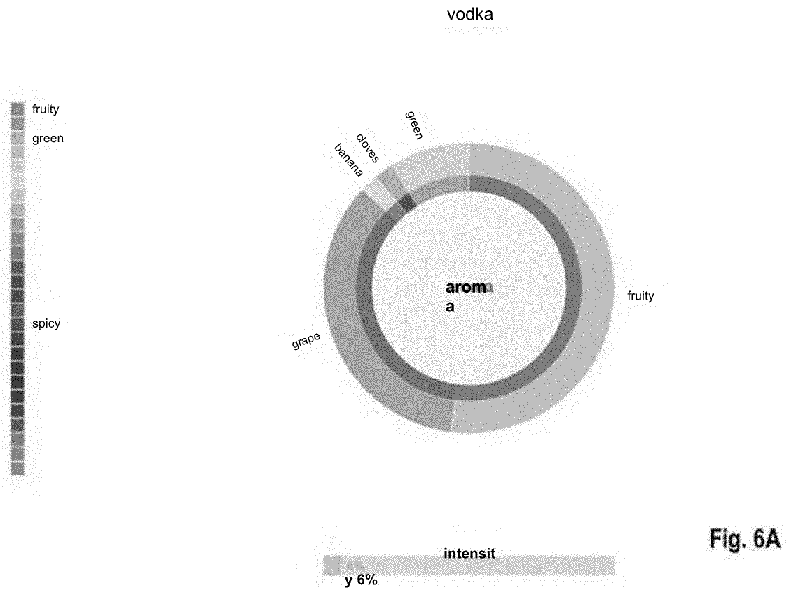

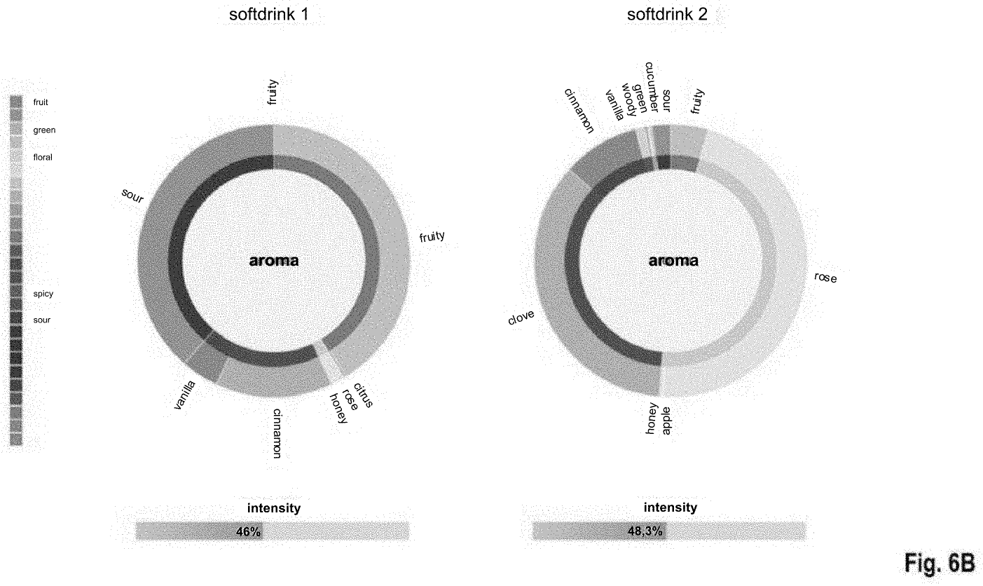

Also provided herein are computer-implemented methods and systems for providing sensory information of ingredients, phases and/or dishes as such to interested individuals. In some embodiments, the computer-implemented methods and systems are used as educational or search tools, for example, in a culinary teaching environment or generally to interested individuals. In some embodiments, the sensory parameters of any ingredient, phase and/or dish can be presented in one form or another to an interested individual. In some embodiments, the presentation forms include but are not limited to a visualization presentation (such as photo, video or animation), a text presentation, an audio presentation, or a combination thereof. A non-limiting exemplary presentation form is shown in FIG. 6A, when, for example, a user is interested in the aroma profile of vodka. Here, the multiple component aroma profile is presented in a manner such that the relative strength or intensity of each component is reflected by visual indictors; for example, the strength of intensity of a particular type of aroma is proportional to the length of the curve representing the aroma.

In some embodiments, differences between the sensory parameters of two or more ingredients, phases and/or dishes can be presented in one form or another to an interested individual. A non-limiting exemplary presentation form is shown in FIG. 6B. In some embodiments, the presentation of sensory parameters can be grouped per sensory modality. In FIG. 6B, the differences in aroma profiles of two different ingredient (or phase in a dish) are readily visible through color coding and graphic representation.

Also provided herein are computer-implemented methods and systems for screening an existing knowledge database of ingredients, phases and/or dishes based on one or more sensory parameters in order to generate a list of ingredients, phases and/or dishes containing the screened sensory parameters as such. Such information is stored in one or more database organized according to sensory modalities. For example, a user is interested in finding ingredients that are sweet, sour or fruity can look up for ingredients within such categories.

Also provided herein are computer program products that can be used on any suitable device, including but not limited to a networked device, a local device, a vending machine, a food dispensing machine, a drink dispensing machine, an automated drink maker, an automated cocktail maker, an automated food preparation machine, a desktop computer, a laptop computer, a mobile device, a handheld device, a tablet, an iPad, a Kindle, a cellular phone, a smart phone, a personal digital assistant (PDA), a networked television, a networked media player, and a networked digital video recorder (DVR).

### Systems and Devices

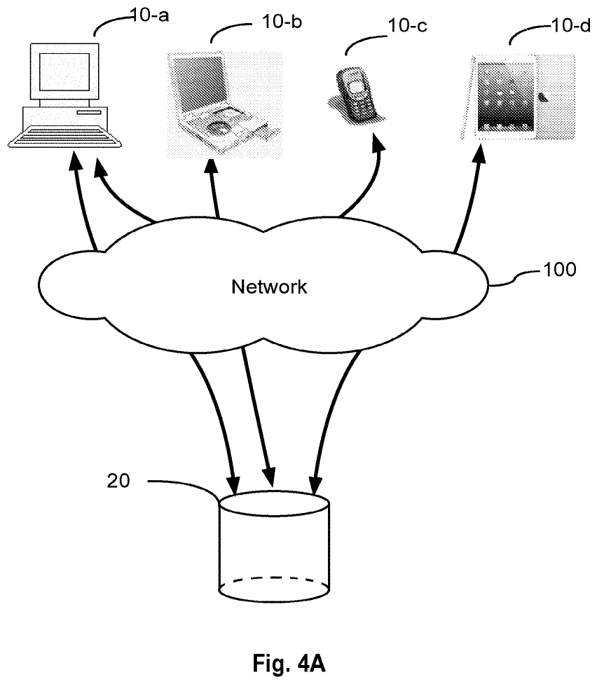

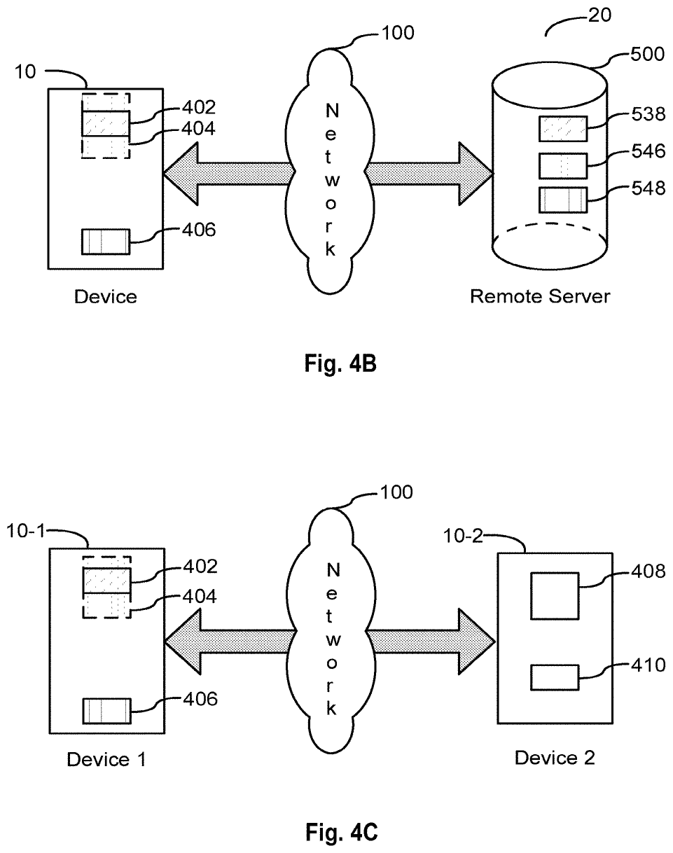

Also provided herein are systems and devices for implementing the methods for substituting one or more ingredients or an entire dish in a food or drink recipe as described herein. As illustrated in FIG. 4A, computer device 10 are connected to a remote data server 20 via network 100. A computer device includes but is not limited to, for example, a networked device, a local device, a desktop computer, a laptop computer, a mobile device, a handheld device, a tablet, an iPad, a Kindle, a cellular phone, a smart phone, a personal digital assistant (PDA), a networked television, a networked media player, or a networked digital video recorder (DVR).

In one aspect, a user can start substituting an ingredient in a food or drink recipe or the entire food or drink recipe, for example, by launching a network-based interface through a host application on the computer device. Computer device 10 connects to a remote server 20 via network 100.

In some embodiments, the host application (e.g., 402 of FIG. 4B) is an embedded application in another program (e.g., as part of a web interface such as a browser). In some embodiments, a host application is a stand-alone program; for example, a mobile app or a dedicated computer program run on a tablet, a laptop or desktop computer. In some embodiments, the user sends one or more keywords (e.g., a target ingredient or a target dish or recipe) via the interface. The keywords can be processed by various tools/programs on the remote database (e.g., data processing application 538, network application 546, and customer support tools 548). Processing results, e.g., one or more substitute ingredients, one or more substitute dishes, one or more modified recipes based on the substitute ingredients, one or more cooking/processing techniques, and etc. are sent to various computer devices 10.

In some embodiments, a network browser, often a web browser, is a software application that enables a user to display and interact with text, images, videos, music and other information typically located on a Web page at a website on the World Wide Web or a local area network. For example, as depicted in FIGS. 4B and 4C, client device 10 comprises a host application 402 that is embedded in a network browser 404 while client device 10 comprises a host application 402. In some embodiments, host application 402 may be an Application Programming Interface (API) or an Application Binary Interface (ABI) application embedded in a network browser 404, for example, Internet Explorer, Mozilla Firefox, Safari, Opera, Opera Mini, Camino, Netscape, or Lynx.

In some embodiments, computer devices 10 are equipped with network capacity (e.g., through a network module 406 as depicted in FIGS. 4B and 4C). In some embodiments, network module 406 allows the client devices to communicate across different network platform.

In some embodiments, computer device 10 connects to another computer device via network 100. For example, one of the computer devices (e.g., device 2 in FIG. 4C) can serve as a local host of database (e.g., elements 408) and tools (e.g., element 410) for processing requests and sending results to the other device. In some embodiments, a local host such as device 2 functions as an intermediate between a user device (e.g., device 10-1) and a remote data server.

### Remote Data Server

In some embodiments, remote data server 20 comprises a central processing unit 510, a power source 512, a user interface 520, communications circuitry 516, a bus 514, a non-volatile storage controller 526, an optional non-volatile storage 528, and a memory 530.

Memory 530 may comprise volatile and non-volatile storage units, for example random-access memory (RAM), read-only memory (ROM), flash memory and the like. In some embodiments, memory 530 comprises high-speed RAM for storing system control programs, data, and application programs, e.g., programs and data loaded from non-volatile storage 528. It will be appreciated that at any given time, all or a portion of any of the modules or data structures in memory 530 can, in fact, be stored in memory 528.

User interface 520 may comprise one or more input devices 524, e.g., keyboard, key pad, mouse, scroll wheel, and the like, and a display 522 or other output device. A network interface card or other communication circuitry 516 may provide for connection to any wired or wireless communications network 100 (e.g., FIGS. 4A and 4B). Internal bus 514 provides for interconnection of the aforementioned elements of the remote data server 20.

In some embodiments, operation of remote data server 20 is controlled primarily by operating system 532, which is executed by central processing unit 510. Operating system 532 can be stored in system memory 530. In addition to operating system 532, a typical implementation of system memory 530 may include a file system 534 for controlling access to the various files and data structures used by the present invention, one or more application modules 336, and one or more databases or data modules 550.

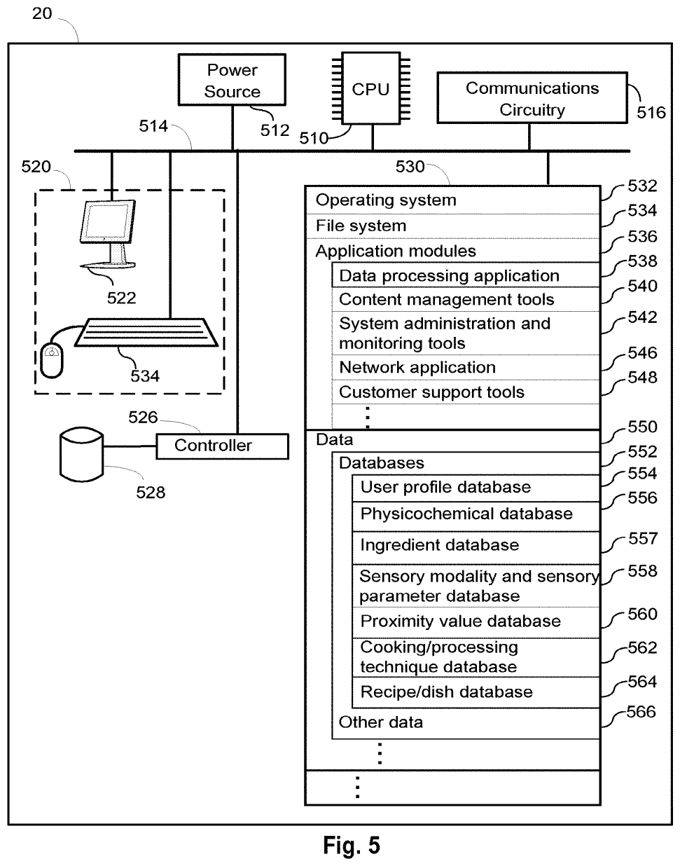

In some embodiments in accordance with the present invention, applications modules 336 may comprise one or more of the following modules described below and illustrated in FIG. 5.

Data processing application 538. In some embodiments, a data processing application 538 receives and processes content shared between client devices 10 and between a client device 10 and remote data server 20. For example, manually entered or selected data (e.g., target ingredient, sensory parameters, ingredients and etc.) are sent from client devices 10 to remote data server 20 and subsequently stored by remote data server 20.

By applying computation techniques (e.g., hash functions), data processing application 538 turns raw data sent from a client device into digital data to construct one or more databases. For example, most frequently entered ingredients are used to rank and organize ingredients into an ingredient database or a database of ingredient types (e.g., element 558 or 558-a).

In some embodiments, data processing application 538 is used to compute a proximity value between two ingredients (or two dishes) that are being compared. In some embodiments, data processing application 538 is used to compare and rank proximity values from multiple pairs of ingredients or dishes. In some embodiments, data processing application 538 is used to compute an intensity value for each sensory modality of an ingredient and subsequently ingredients in a phase of a dish. In some embodiments, data processing application 538 is used to compute quantities of each ingredient in a final dish.

Content management tools 540. In some embodiments, content management tools 540 are used to organize different forms of databases 552 into multiple databases, e.g., a user profile database 554, a physicochemical characteristics database 556, an ingredient database 558, a proximity value or distance value database 560, a preparation method database 562, a recipe or dish database 564 and other data 566. In some embodiments in accordance with the present invention, content management tools 540 are used to search and compare any of the databases hosted on remote data server 20. Content in accordance with the present invention may be, for example, a text message, a URL, a web link, a note message, a post message, a file, an image, an audio file, a video file, a flash file, a media file, a slideshow file, any printable file, or any ASCII or binary file or data structure.

The databases stored on remote data server 20 comprise any form of data storage system including, but not limited to, a flat file, a relational database (SQL), and an on-line analytical processing (OLAP) database (MDX and/or variants thereof). In some specific embodiments, the databases are hierarchical OLAP cubes. In some embodiments, the databases each have a star schema that is not stored as a cube but has dimension tables that define hierarchy. Still further, in some embodiments, the databases have hierarchy that is not explicitly broken out in the underlying database or database schema (e.g., dimension tables are not hierarchically arranged). In some embodiments, the databases in fact are not hosted on remote data server 20 but are in fact accessed by centralized data server through a secure network interface. In such embodiments, security measures such as encryption is taken to secure the sensitive information stored in such databases.

System administration and monitoring tools 542. In some embodiments in accordance with the present invention, the system administration and monitoring tools 542 administer and monitor all applications and data files of remote data server 20. System administration and monitoring tools 542 control which servers or devices have access to remote data server 20. In some embodiments, security administration and monitoring is achieved by restricting data download access from remote data server 20 such that the data is protected against malicious access. In some embodiments, system administration and monitoring tools 542 use more than one security measure to protect the data stored on remote data server 20. In some embodiments, a random rotational security system may be applied to safeguard the data stored on remote data server 20. In some embodiments, a user gains access to a database on remote data server 20 using a user account via a password. In such embodiments, a user profile can be established to keep track of the activities of the user and the recipes created by the user.

Network application 546. In some embodiments, network applications 546 connect a remote data server 20 to multiple network services. In some embodiments, a remote data server 20 is connected to multiple types of client devices 10, which requires that remote data server 20 be adapted to communication with different types of network interfaces, for example, router based computer network interfaces, switch based phone like network interfaces, and cell tower based cell phone wireless network interfaces. In some embodiments in accordance with the present invention, upon recognition, a network application 546 receives data from intermediary gateway servers before it transfers the data to other application modules such as data processing application 538, content management tools 540, and system administration and monitoring tools 542.

Customer support tools 548. Customer support tools 548 assist users with information or questions regarding their accounts, technical support, billing, and etc. In some embodiments, customer support tools 548 may allow a member to manually input or select the member's interest category to facilitate better characterization of the member's sharing preference profile.

In some embodiments, each of the data structures stored on the remote data server 20 is a single data structure. In other embodiments, any or all such data structures may comprise a plurality of data structures (e.g., databases, files, and archives) that may or may not all be stored on remote data server 20. The one or more data modules 550 may include any number of databases 552 organized into different structures (or other forms of data structures) by content management tools 540.

In addition to the above-identified modules, data 550 may be stored on remote data server 20 or on a computer that is addressable by remote data server (e.g., any computer that the remote data server can send information to and/or retrieve information from). Such data comprises content databases 552 and recipe or dish data 564. Exemplary databases 552 include, but are not limited to, user profile database 554, physiochemical characteristics database 556, ingredient database 558, proximity value or distance value 560, preparation method dataset 562, and recipe or dish database 564 and other data 566, which are described below in more detail.

User profile database 554. In some embodiments, remote data server 20 hosts a user profile database 554. In some embodiments, user profile database 554 comprises information concerning a particular user; for example, the user's preferences and allergies. In some embodiments, a user's search histories are used to improve or optimize his or her profile. In some embodiments, user profile database 554 is stored on, and managed by, programs of remote data server 20. In some embodiments of the present invention, user profile database 554 may be searched by a data processing application 538. In some embodiments of the present invention, user profile database 554 may be maintained, updated and managed by content management tools 540. In some embodiments, each time a new target ingredient is inputted by the member, user profile database 554 will also be updated accordingly.

Physicochemical characteristics database 556. In some embodiments, remote data server 20 hosts a physicochemical characteristics database 556. One or more physicochemical characteristics associated with ingredients or dishes (e.g., phases of a dish) are organized and stored in physicochemical characteristics database 556. In some embodiments, physicochemical characteristics of an ingredient or a dish are converted to sensory data; for example, one or more sensory parameters representing an ingredient or a phase in dish. In some embodiments, the sensory parameters for a particular sensory modality among a plurality of sensory modalities of an ingredient are stronger than sensory parameters for other sensory modalities. In some embodiments, two ingredients can affect the sensory modality of each other. For example, the effect of spiciness can be attenuated by sweetness. In some embodiments, individual components from a pair of ingredients are compared before a compatibility score is calculated. In some embodiments, an additional parameter (e.g., a weight or intensity parameter) is added to the sensory parameters to reflect the strength of each sensory modality. In some embodiments, an additional parameter (e.g., a weight or intensity parameter) is added to the phase sensory parameters to reflect the strength of each phase.

Ingredient database 557. In some embodiments, remote data server 20 hosts an ingredient database 558. One or more characteristics associated with an ingredient are organized and stored in ingredient database 558. In some embodiments, ingredient database 558 is organized according to sensory modalities. In some embodiments, ingredient database 558 is organized by types of food or drink associated with the ingredients. In some embodiments, ingredient database 558 includes information concerning the molecular components of the individual ingredients, including but not limited to physicochemical data thereof and sensory modalities and/or sensory parameters associated therewith.

Sensory modality and sensory parameter database 558. In some embodiments, sensory modalities and sensory parameters are organized independent from ingredients, dishes or recipes. Instead, sensory modalities and sensory parameters are organized according to the specific molecular components responsible for the sensory modalities and sensory parameters. In some embodiments, threshold values are used to eliminate molecular components that are in quantities too small to trigger sensory response from a user.

Proximity value or distance value database 560. In some embodiments, remote data server 20 hosts a proximity value or distance database 560. Also, included in the data are various types of proximities and distances.

Preparation method database 562. In some embodiments, a preparation method database 562 is stored on remote data server 20. Methods of preparation include but are not limited to methods of cooking, processing, assembling, making, and etc. Exemplary preparation methods include but are not limited to being shaken, being stirred, gassing, de-gassing baking, frying, steaming, roasting, hot processing, cold processing, marinating, salting, curing, pureeing, chopping, kneading, blending, grinding, poaching and etc. In some embodiments, preparation methods are not organized in an independent database, but instead, as components of a recipe or dish, organized in recipe or dish database 564.

Recipe or dish database 564. In some embodiments, a recipe or dish database 564 is stored on remote data server 20. In some embodiments, recipes created by users are stored in recipe or dish database 564. In some embodiments, the recipes are organized by food types, drink types, cuisine types, cooking techniques, processing types, and ingredient types. A user can choose to access recipes presented to the user by the system in the past. A user can also choose to share recipes with other users through a community-based sharing interface, e.g., by customer support tools 548.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Ingredient Substitution Based on Taste

This example illustrates that a food or drink ingredient can be substituted or replaced by another food or drink ingredient with similar taste.

At the initial input step, an ingredient was selected from a list of ingredients. Here the target ingredient is cauliflower.

Using the process for ingredient substitution described herein, taste distance towards the taste parameters of cauliflower was calculated to produce the following list of replacement or substitute ingredients for cauliflower. The smaller the distance, the higher the suitability of the substitution ingredient.

| Ingredients | Taste distance |
|---|---|
| Almond | 1.629824 |
| Anise | 1.535975 |
| Apple | 0.24568 |
| Apricot | 0.278222 |
| Artichoke | 0.210442 |
| Asparagus | 0.091257 |
| Banana | 0.389385 |
| Basil | 0.092506 |
| Beans raw | 0.315443 |
| Beef grilles | 0.367585 |
| Beetroot | 0.180215 |
| Beetroot raw | 0.196019 |
| Bell pepper | 0.096958 |
| Bergamot | 1.444509 |
| Bilberry | 0.207854 |
| Bitter orange peel | 1.432446 |
| Black currant | 0.207854 |
| Blueberry | 0.243443 |
| Bread | 0.988819 |
| Broccoli | 0.078224 |
| Buckwheat | 1.510781 |
| Butter | 1.470672 |
| Buttermilk | 0.116693 |
| Cabbage Chinese | 0.100118 |
| Cabbage cooked | 0.051443 |
| Cabbage raw | 0.047705 |
| Calamus | 0.092506 |
| Caraway | 1.53331 |
| Caraway seed | 1.543604 |
| Cardamom | 2.154791 |
| Carrot | 0.086553 |
| Cauliflower cooked | 0.029941 |
| Celery leaves | 0.073214 |
| Celery root | 0.064694 |
| Cheese blue | 1.095006 |
| Cheese Brie | 0.892519 |
| Cheese camembert | 0.892519 |
| Cheese cheddar | 1.144626 |
| . . . | |
| (list truncated for | |
| presentation) | |

In this example, the output substitute ingredient list was presented according to the alphabetic order of the ingredients. The user can also choose to output substitute ingredient list according to the value of the taste distance score.

Based on their respective taste distance values in the initial output substitute ingredient list, a taste distance ranked subset of candidate substitution ingredients was generated.

| Ingredients | Taste distance |
|---|---|
| Broccoli | 0.078224 |
| Cabbage cooked | 0.051443 |
| Cabbage raw | 0.047705 |
| Carrot | 0.086553 |
| Cauliflower cooked | 0.029941 |
| Celery leaves | 0.073214 |
| Celery root | 0.064694 |
| Chicory | 0.087986 |
| Cucumber | 0.080012 |
| Egg plant | 0.047234 |
| Endives | 0.084437 |
| Fennel sweet | 0.05431 |
| Kale | 0.073741 |
| Lamb lettuce | 0.087862 |
| Leek raw | 0.069176 |
| Lettuce | 0.051854 |
| Loquat | 0.073107 |
| Mushroom | 0.062795 |
| Parsley leaves | 0.064846 |
| Turnip | 0.068914 |

### Example 2

### Ingredient Substitution Based on Aroma

This example illustrates that a food or drink ingredient can be substituted or replaced by another food or drink ingredient with similar aroma. Here, cauliflower was used the target ingredient. The subset of substitute ingredients from Example 1 is listed below, showing aroma distance values in addition to taste distance values.

| Ingredients | Taste distance | Aroma distance |
|---|---|---|
| Broccoli | 0.078224 | 1.903752 |
| Cabbage cooked | 0.051443 | 1.292977 |
| Cabbage raw | 0.047705 | 1.905128 |
| Carrot | 0.086553 | 2.265201 |
| Kale | 0.029941 | 1.897821 |
| Celery leaves | 0.073214 | 2.091182 |
| Celery root | 0.064694 | 1.945327 |
| Chicory | 0.087986 | 2.082295 |
| Cucumber | 0.080012 | 1.933091 |
| Egg plant | 0.047234 | 1.893821 |
| Endives | 0.084437 | 1.920747 |
| Fennel sweet | 0.05431 | 2.033089 |
| Cauliflower cooked | 0.073741 | 0.084911 |
| Lamb lettuce | 0.087862 | 1.898035 |
| Leek raw | 0.069176 | 1.987701 |
| Lettuce | 0.051854 | 1.710377 |
| Loquat | 0.073107 | 1.917572 |
| Mushroom | 0.062795 | 1.923213 |
| Parsley leaves | 0.064846 | 1.921876 |
| Turnip | 0.068914 | 2.113416 |

Based on their respective aroma distance values in the initial output substitute ingredient list, an aroma distance ranked subset of candidate substitution ingredients is outputted to the user.

| Ingredients | Taste distance | Aroma distance |
|---|---|---|
| Cauliflower cooked | 0.073741 | 0.084911 |
| Cabbage cooked | 0.051443 | 1.292977 |
| Lettuce | 0.051854 | 1.710377 |
| Egg plant | 0.047234 | 1.893821 |
| Kale | 0.029941 | 1.897821 |
| Lamb lettuce | 0.087862 | 1.898035 |
| Broccoli | 0.078224 | 1.903752 |
| Cabbage raw | 0.047705 | 1.905128 |
| Loquat | 0.073107 | 1.917572 |
| Endives | 0.084437 | 1.920747 |
| Parsley leaves | 0.064846 | 1.921876 |
| Mushroom | 0.062795 | 1.923213 |
| Cucumber | 0.080012 | 1.933091 |
| Celery root | 0.064694 | 1.945327 |
| Leek raw | 0.069176 | 1.987701 |
| Fennel sweet | 0.05431 | 2.033089 |
| Chicory | 0.087986 | 2.082295 |
| Celery leaves | 0.073214 | 2.091182 |
| Turnip | 0.068914 | 2.113416 |
| Carrot | 0.086553 | 2.265201 |

### Example 3

### Ingredient Substitution Based on Aroma and Taste

This example illustrates that a replacement or substitution distance can be calculated based on taste and aroma distance values.

Here, taste distance values and aroma distance values were combined into a global substitution distance value, taking into account the relative importance of taste parameters vs aroma parameters in the target ingredient (when target ingredient has a relative strong aroma intensity opposed to taste intensity, aroma distance will be weighed harder in the global distance values, and vice versa). The subset of candidate substitution ingredients are ranked according to their global distance value and presented to the user.

| | Taste | Aroma | Substitution |
|---|---|---|---|
| Ingredients | distance | distance | distance |
| Cauliflower cooked | 0.073741 | 0.084911 | 0.02702 |
| Cabbage cooked | 0.051443 | 1.292977 | 0.351265 |
| Kale | 0.029941 | 1.897821 | 0.406505 |
| Lettuce | 0.051854 | 1.710377 | 0.466286 |
| Egg plant | 0.047234 | 1.893821 | 0.495565 |
| Cabbage raw | 0.047705 | 1.905128 | 0.5007 |
| Fennel sweet | 0.05431 | 2.033089 | 0.565643 |
| Mushroom | 0.062795 | 1.923213 | 0.570299 |
| Parsley leaves | 0.064846 | 1.921876 | 0.578006 |
| Celery root | 0.064694 | 1.945327 | 0.584454 |
| Loquat | 0.073107 | 1.917572 | 0.607892 |
| Leek raw | 0.069176 | 1.987701 | 0.615015 |
| Broccoli | 0.078224 | 1.903752 | 0.621714 |
| Cucumber | 0.080012 | 1.933091 | 0.63759 |
| Endives | 0.084437 | 1.920747 | 0.648673 |
| Lamb lettuce | 0.087862 | 1.898035 | 0.652296 |
| Turnip | 0.068914 | 2.113416 | 0.652823 |
| Celery leaves | 0.073214 | 2.091182 | 0.663356 |
| Chicory | 0.087986 | 2.082295 | 0.716065 |
| Carrot | 0.086553 | 2.265201 | 0.773366 |

### Example 4

### Ingredient Substitution Based on Texture

This example illustrates that a food or drink ingredient can be substituted or replaced by another food or drink ingredient with similar texture. Here, whipped cream is used as the query ingredient.

| Ingredients | Texture distance |
|---|---|
| chocolate mousse texture | 0.714413 |
| pastry cream texture | 0.990323 |
| mayonaise texture | 1.122969 |
| sherbet texture | 1.174393 |
| marshmallow texture | 1.213432 |
| gelatin gel texture | 1.401956 |
| cream sauce texture | 1.416675 |
| butter texture | 1.492308 |
| yoghurt texture | 1.521271 |
| mashed potato texture | 1.547342 |
| dark chocolate texture | 1.560943 |
| oil texture | 1.67714 |
| toffee texture | 1.767584 |
| aires texture | 1.776226 |
| quatre quart texture | 1.78198 |
| bread crumb texture | 1.88547 |
| chocolate chip cookie texture | 1.946975 |
| ripe peach texture | 1.959957 |
| champagne texture | 1.98699 |
| sponge cake texture | 1.989357 |
| jam texture | 2.031248 |
| steak medium rare internal texture | 2.046965 |
| hot sauce texture | 2.0746 |
| water texture | 2.076672 |
| meringue texture | 2.077972 |
| hard cheese texture | 2.080536 |
| granita texture | 2.133029 |
| French fries external texture | 2.136412 |
| vodka texture | 2.147456 |
| Twix biscuit texture | 2.239077 |
| whole hazelnut texture | 2.298362 |
| green peppercorn texture | 2.333908 |
| butter crisp texture | 2.399612 |
| potato chips | 2.439655 |
| sugar tuile texture | 2.482781 |
| castor sugar texture | 2.572988 |
| grated coconut texture | 4.377482 |

### Example 5

### Dish Substitution

Consider following recipe (hereinafter referred to as Recipe81).
200 g Chavroux
15 g Sugar syrup
2 g Gelatin leaf
100 g Cream
25 g White sugar

The ingredients above were prepared first based on these steps of preparation: soak the gelatin leaves in cold water; dissolving in the sugar syrup and mix with the Chavroux; whipping the cream with the sugar; mixing 80 g whipped cream with the Chavroux; and placing in the refrigerator until use.
150 g Passion fruit juice
50 g White sugar
1.7 g Agar

The ingredients above were then prepared based on these steps of preparation: blending the passion fruit juice with the sugar and the agar; bringing to a boil; pouring a thin layer on a plate and let gel; cutting into equal sized bands and removing every second band.
100 g Water
100 g Cub-o-cream
1.7 g Agar

The ingredients above were prepared first based on these steps of preparation: mixing the ingredients and bring to boil; allow to cool; pouring some cub-o-creme gel between the bands of passion fruit gel; letting the mixture gelatinize; cutting into rectangles; piping some Chavroux crème on a rectangle and rolling into a cannelloni.
75 g Wheat flour
25 g Pistachio nuts
60 g White sugar
60 g Butter

The ingredients above were mixed into a crumble and baked in an oven at 100° C. The resulting mixture is allowed to cool.
100 g Raspberry puree
45 g Cub-o-cream
10 g White sugar
2 Egg
50 g Butter

The ingredients above were processed as follows. The unsweetened raspberry coulis was mixed with the sugar, the cub-o-cream and the eggs. The mixture was brought to a boil and then allowed to cool to 37° C. Butter was then added and mixed.

This dessert “recipe81” should be viewed as a collection of homogenic subsystems, i.e., phases, with each a set of dimensions. Following phases were observed respectively in recipe81:
1. bavarois
2. agar gel
3. crumble
4. emulsion

The taste and aroma of each phase in this recipe can easily be calculated based on the sensorial perception of its participating ingredients, their respective proportions and preparation methods. However, the texture modality does not follow the assumption of summability, yet a series of textural parameters can be quantified based on recipe insight, sensorial analysis and/or instrumental analysis.

The ideal alternative dish would be a dish were all its phases are pairwise ideal substitution phases of the phases of the recipe81. Alternative phases for the bavarois phase are searched in a recipe database, based on its taste, aroma and texture parameters. Global distances for each phase in the database are kept. This action is repeated for each phase in recipe81. The global distances for each phase of recipe81 towards all phases in the database are combined per database dish to obtain a final global distance towards the target recipe for each database recipe.

Following this strategy, recipe380 recipe can be found as a suitable alternative of recipe81 (hereinafter referred to as Recipe380).

Speculoos Crumble
125 g White sugar
125 g Refined cane sugar
125 g Butter
125 g Aristo Primeur Cake
250 g Wheat flour
250 g PatisFrance Amandes Blanchies Poudre
5 g Quatre épices

Mix all the ingredients in a bowl with a flat beater at slow speed. When the pieces start to form lumps, spread them out on a baking tray and place them in the freezer. Bake at +/−160° C. for 16 minutes.

Marzipan Biscuit
240 g Marzipan
240 g Egg
45 g Wheat flour
3 g Baking powder
37.5 g Butter
37.5 g Aristo Primeur Cake

Mix the marzipan and add the eggs bit by bit. Add the flour and the baking powder. Finally add the butter and the Aristo Primeur Cake. Spread out on a baking tray and bake at 200° C. for 10 min.

Lavender Mousse
160 g Milk
5 g Lavender
6 g Gelatin leaf
300 g Blanc Dominican Republic 31, Belcolade Origins
150 g Cream, half whipped
150 g Festipak

Heat up the milk with the lavender. Let it infuse for 10 min. Pre-soak the gelatin. Sieve the milk and pour onto the Belcolade Origins Dominican Republic 31. Mix to make it homogeneous. When the ganache reaches 35° C. add the half whipped cream (fresh cream & Festipak). Pipe in cylinders of 5 cm. Freeze. After freezing finish with white velvet pipe.

Apricot Balls
500 g PatisFrance Starfruit Apricot
80 g Glucose syrup
40 g White sugar
12 g Gelatin leaf

Boil the PatisFrance Starfruit Apricot with the glucose and the sugar. Add the pre-soaked gelatin leaves. Pipe in demi-sphere moulds, diameter 3 cm, and freeze.

Lavender Foam
250 g Milk
50 g White sugar
1 g Lavender
2 g Soy lecithin

Heat up the whole milk with the sugar and let the lavender infuse. Add the lecithin. Place in a bowl and cover with a cling film and put it in the fridge.

Cut the marzipan biscuit 5×5 cm. Place the cylinder of the lavender mousse on top. Decorate with the Speculoos crumble, apricot balls and lavender foam.

The lavender mousse is related to Chavroux Bavarois, furthermore both recipe have crumbles of similar taste and texture. The apricot fluid gel in recipe380 is comparable to the raspberry emulsion of recipe81.

It can be observed that modality weights in the calculation of the global distance can differ from the ingredient substitution algorithm, especially when texture is considered. It can be reasoned that ingredient substitution happens before dish preparation, thus the texture can be altered by the blessing of the user, therefore the texture modality might be considered relatively less important opposed to taste and aroma distance. This is not the case when searching for dish alternative, which already stipulate a final set of textures

### Example 6

### Cocktail Sensory Parameters Calculation

In this example, the calculation of the sensory parameters of a Margarita cocktail is examined. A standard recipe for Margarita include the following ingredients at respective volumes (cl: centiliter):
3.5 cl Tequila
2 cl triple sec
1.5 cl lime juice

The method for preparing the drink is shaken.

First, the sensory parameters of the cocktail are calculated based on its recipe and the dimensions of the ingredients.

| Quantity | Unit | Ingr_name | Prop | TEX1 | TEX2 | TASTE1 | TASTE2 | TASTE3 |
|---|---|---|---|---|---|---|---|---|
| 3.5 | cl | Tequila | 0.47 | 30.5 | 66.9 | 0.1 | 0 | 0 |
| 2 | cl | Triple sec | 0.31 | 23 | 44 | 32.13 | 2.65 | 0 |
| 1.5 | cl | lime juice | 0.21 | 0 | 89.4 | 1.15 | 47.6 | 0 |

Performing sumproduct give dimensions of the cocktail before preparation:

| Title | TEX1 | TEX2 | TASTE1 | TASTE2 | TASTE3 |
|---|---|---|---|---|---|
| MARGARITA | 21.58 | 64.53 | 10.41 | 11.07 | 0 |

Applying a transformation for preparation method gives follow sensory parameters for the cocktail

| Title | TEX1 | TEX2 | TASTE1 | TASTE2 | TASTE3 |
|---|---|---|---|---|---|
| MARGARITA | 13.00 | 78.63 | 6.27 | 6.67 | 0 |

### Example 7

### Cocktail Substitution

This example illustrates that an alternative cocktail with similar sensory properties can be generated based on a target cocktail.

At the initial input step, a cocktail was selected from a list of cocktails. Here the target cocktail is a Daiquiri.

Using the process for ingredient substitution described herein, taste distance towards the taste parameters of a Daiquiri was calculated to produce a filtered list of substitute cocktails for a Daiquiri.

Next, aroma distance towards the aroma parameters of daiquiri is calculated for the subset of alternative cocktails.

Next, taste distance values and Aroma distance values are combined into a global distance value, taking into account the relative importance of taste parameters vs aroma parameters in the inputted cocktail, daiquiri. The subset of substitution cocktails are ranked according to their global distance value and presented to the user. The smaller the global distance, the higher the suitability of the substitution ingredient

| Substitute cocktails | Global dist. | Aroma dist. | Taste dist. |
|---|---|---|---|
| Daiquiri | 0 | 0 | 0 |
| Bacardi | 0.000321 | 0.01568 | 0.253883 |
| Whiskey sour | 0.001004 | 0.424984 | 0.118517 |
| Bramble | 0.001424 | 0.269112 | 0.157528 |
| White lady | 0.001542 | 0.286378 | 0.158484 |
| Caipirinha | 0.001669 | 0.038883 | 0.329484 |
| Sidecar | 0.001769 | 0.136703 | 0.215882 |
| Kamikaze | 0.002594 | 0.34347 | 0.178564 |
| Yellow bird | 0.002614 | 0.080198 | 0.29902 |
| Clover club | 0.004434 | 0.257296 | 0.238845 |
| Russian spring punch | 0.004498 | 0.425226 | 0.20108 |
| Pisco sour | 0.005204 | 0.238268 | 0.259668 |
| Mai-tai | 0.006501 | 0.092524 | 0.392052 |

### Example 8

### Filtering a Cocktail Database on an Ingredient and its Substitute Ingredients

This example illustrates that a cocktail database screening on ingredients can deliver improved results when substitution ingredients of the query ingredients are supplemented to the query.

In this example a user wants to browse cocktails containing whisky and lemon juice as ingredients.

A resulting list of suitable substitution ingredients for a target ingredient can be used to screen a knowledge database of recipes for recipes primarily containing the target ingredient, secondarily containing any of the suitable substitution ingredient. This embodiment can become increasingly interesting when applying extended ingredient search queries on a knowledge database of recipes. Such an embodiment can also be combined with the process for dish substitution.

The following table illustrates Whisky alternatives.

| Substitute ingredients | Global dist. | Taste dist. | Aroma dist. |
|---|---|---|---|
| whiskey | 0 | 0 | 0 |
| eau de vie | 0.000744 | 0.004472 | 0.163755 |
| genever | 0.001243 | 0.004472 | 0.273852 |
| gin | 0.001243 | 0.004472 | 0.273852 |
| schnapps | 0.001692 | 0.010198 | 0.163755 |
| grappa | 0.001876 | 0.010198 | 0.181634 |
| rum | 0.002114 | 0.005657 | 0.368359 |
| pisco | 0.002778 | 0.010198 | 0.268891 |
| pastis | 0.003054 | 0.007211 | 0.417677 |
| ouzo | 0.003054 | 0.007211 | 0.417677 |
| kirsch | 0.003928 | 0.010198 | 0.380229 |
| aquavit | 0.004315 | 0.010198 | 0.417677 |
| Tequila | 0.004873 | 0.010198 | 0.471725 |
| Metaxa | 0.006312 | 0.034525 | 0.181109 |
| Calvados | 0.006312 | 0.034525 | 0.181109 |
| applejack | 0.006312 | 0.034525 | 0.181109 |
| Armagnac | 0.013385 | 0.034525 | 0.384041 |
| Cognac | 0.013385 | 0.034525 | 0.384041 |
| brandy | 0.013385 | 0.034525 | 0.384041 |
| Pernod | 0.014558 | 0.034525 | 0.417677 |

The following table illustrates lemon juice alternatives.

| Substitute ingredients | Global dist. | Taste dist. | Aroma dist. |
|---|---|---|---|
| lemon juice | 0 | 0 | 0 |
| lime juice | 9.09E−05 | 0.067119 | 0.232652 |
| passion fruit juice | 0.016882 | 0.317714 | 0.472116 |
| passion fruit | 0.016882 | 0.317714 | 0.472116 |
| ginger ale | 0.144931 | 0.762264 | 0.318902 |
| 7-Up | 0.159034 | 0.762363 | 0.349802 |
| cranberry juice | 0.176977 | 0.76231 | 0.389345 |
| peach | 0.209958 | 0.781614 | 0.429537 |
| blood orange juice | 0.228955 | 0.774558 | 0.480903 |
| pineapple juice | 0.234516 | 0.784695 | 0.474325 |
| Cola | 0.237509 | 0.762264 | 0.522607 |
| tomato juice | 0.250355 | 0.826494 | 0.435495 |
| Papaya juice | 0.255995 | 0.840085 | 0.424698 |
| cherry | 0.256796 | 0.769932 | 0.548851 |
| orange juice | 0.266368 | 0.815983 | 0.480903 |
| cider | 0.281314 | 0.875984 | 0.41328 |
| apple juice | 0.288112 | 0.880237 | 0.417355 |
| vinegar | 34899.34 | 0.315068 | 1000000 |

Next these two lists of substitution ingredients are matched in a cocktail database and global distances are used to score each matched cocktail. This results in a list of cocktail recommendations with on top cocktails with both whisky and lemon juice, further down cocktails recommendations that contain an alternative of the desired ingredients.

The main difference with classic recipe filtering techniques on ingredients is that in this case there is no categorical drop-off. For example, cocktails with lime juice will be fairly high on top of the list since lime juice is a good alternative of lemon juice. When using classic techniques e.g., those available at www<dot>cocktaildb<dot>com</>index, cocktails with lime juice would not show at all since they do not contain the desired ingredient. Our recommendations are therefore less strict, more intuitive, more continuous in terms of drop off, and makes intensive database works (classifying ingredients etc.) obsolete.

| | Substitute | Substitute | |
|---|---|---|---|
| | proximity ingr. | proximity ingr. | Combined |
| Row labels | 1 (whisky) | 2 (lemon) | proximity |
| Whiskey sour | 100000 | 100000 | 300000 |
| Long island iced tea | 0.247592 | 100000 | 100157.6 |
| Gin fizz | 0.228478 | 100000 | 100151.4 |
| French 75 | 0.228478 | 100000 | 100151.4 |
| Bramble | 0.228478 | 100000 | 100151.4 |
| Aviation | 0.228478 | 100000 | 100151.4 |
| Casino | 0.228478 | 100000 | 100151.4 |
| White lady | 0.228478 | 100000 | 100151.4 |
| Clover club | 0.228478 | 100000 | 100151.4 |
| John collins | 0.228478 | 100000 | 100151.4 |
| Between the sheets | 0.018787 | 100000 | 100043.4 |
| Planter's punch | 0.018785 | 100000 | 100043.4 |
| Pisco sour | 0.005067 | 100000 | 100022.5 |
| Sidecar | 2.24e−06 | 100000 | 100000.5 |
| Russian spring punch | 8.5e−36 | 100000 | 100000 |
| Lemon drop martini | 8.5e−36 | 100000 | 100000 |
| God father | 100000 | 0 | 100000 |
| Old fashioned | 100000 | 0 | 100000 |
| Bloody mary | 8.5e−36 | 100000 | 100000 |
| Manhattan | 100000 | 0 | 100000 |
| Mint julep | 100000 | 0 | 100000 |
| Singapore sling | 0.228478 | 3943.853 | 3974.1 |
| Mai-tai | 0.03757 | 3943.853 | 3956.063 |
| Cuba libre | 0.018785 | 3943.853 | 3952.479 |
| Daiquiri | 0.018785 | 3943.853 | 3952.479 |
| Yellow bird | 0.018785 | 3943.853 | 3952.479 |
| Caipirinha | 0.018785 | 3943.853 | 3952.479 |
| Bacardi | 0.018785 | 3943.853 | 3952.479 |
| Hemingway special | 0.018785 | 3943.853 | 3952.479 |
| Tommy's margarita | 0.000329 | 3943.853 | 3944.992 |
| Margarita | 0.000329 | 3943.853 | 3944.992 |
| Moscow mule | 8.5e−36 | 3943.853 | 3943.853 |
| Cosmopolitan | 8.5e−36 | 3943.853 | 3943.853 |
| Kamikaze | 8.5e−36 | 3943.853 | 3943.853 |
| French martini | 2.341106 | 1.41e−12 | 2.341108 |
| Angel face | 0.228571 | 0 | 0.228571 |
| Paradise | 0.228478 | 7.44e−13 | 0.228479 |
| Monkey gland | 0.228478 | 7.44e−13 | 0.228479 |
| Gin & tonic 1:2 | 0.228478 | 0 | 0.228478 |
| Gin & tonic 1:3 | 0.228478 | 0 | 0.228478 |
| Derby | 0.228478 | 0 | 0.228478 |
| Dry martini | 0.228478 | 0 | 0.228478 |
| Gin & tonic 2:3 | 0.228478 | 0 | 0.228478 |
| Negroni | 0.228478 | 0 | 0.228478 |
| Tuxedo | 0.228478 | 0 | 0.228478 |
| Vesper | 0.228478 | 0 | 0.228478 |
| Gin & tonic 1:1 | 0.228478 | 0 | 0.228478 |
| Sex on the beach | 0.054167 | 6.49e−12 | 0.054167 |
| Dark ‘n’ stormy | 0.018785 | 1.56e−11 | 0.018786 |
| Mary pickford | 0.018785 | 1.41e−12 | 0.018785 |
| Rose | 0.000943 | 0 | 0.000943 |
| Tequila sunrise | 0.000329 | 7.44e−13 | 0.000329 |
| Horse's neck | 2.24e−06 | 1.56e−11 | 2.25e−06 |
| Stinger | 2.24e−06 | 0 | 2.24e−06 |
| Sazerac | 2.24e−06 | 0 | 2.24e−06 |
| Champagne cocktail | 2.24e−06 | 0 | 2.24e−06 |
| Porto flip | 2.24e−06 | 0 | 2.24e−06 |
| French connection | 2.24e−06 | 0 | 2.24e−06 |
| Sea breeze | 8.5e−36 | 5.74e−12 | 5.74e−12 |
| Bellini | 0 | 2.45e−12 | 2.45e−12 |
| Harvey wallbanger | 8.5e−36 | 7.44e−13 | 7.44e−13 |
| Screwdriver | 8.5e−36 | 7.44e−13 | 7.44e−13 |
| Mimosa | 0 | 7.44e−13 | 7.44e−13 |
| Black Russian | 8.5e−36 | 0 | 8.5e−36 |
| God mother | 8.5e−36 | 0 | 8.5e−36 |
| Americano | 0 | 0 | 0 |
| Campari soda | 0 | 0 | 0 |
| Kir | 0 | 0 | 0 |
| Spritz veneziano | 0 | 0 | 0 |

### Example 9

### Cocktail Substitution Example with Desired Ingredients

In this example, a combined mechanism based on the processes in Example 7 and Example 8 are used to identify cocktails that are similar to a daiquiri and contain whisky and lemon juice. This combined strategy combines global distance calculation towards a list of cocktail substitutes and cocktail scoring based on ingredient presence as used in example 8.

| | | Desired | |
|---|---|---|---|
| | | ingredient | Global |
| | Substitution | presence | dist. to |
| Substitute cocktail | distance | score | daiquiri |
| IBA WHISKEY SOUR | 0.501001 | 200000 | 0.001004 |
| IBA WHISKEY SOUR | 0.507228 | 200000 | 0.007231 |
| (PETER) | | | |
| IBA BRAMBLE | 1.001412 | 100000.2 | 0.001424 |
| IBA WHITE LADY | 1.00153 | 100000.2 | 0.001542 |
| IBA SIDECAR | 1.001759 | 100000 | 0.001769 |
| IBA CLOVER CLUB | 1.004422 | 100000.2 | 0.004434 |
| IBA RUSSIAN SPRING | 1.004488 | 100000 | 0.004498 |
| PUNCH | | | |
| IBA PISCO SOUR | 1.005194 | 100000 | 0.005204 |
| IBA BETWEEN THE | 1.013393 | 100000 | 0.013404 |
| SHEETS | | | |
| IBA AVIATION | 1.025442 | 100000.2 | 0.025455 |
| IBA GOD FATHER | 1.02912 | 100000 | 0.02913 |
| IBA MINT JULEP | 1.056456 | 100000 | 0.056466 |
| IBA FRENCH 75 | 1.060027 | 100000.2 | 0.060039 |
| IBA LEMON DROP | 1.078707 | 100000 | 0.078717 |
| MARTINI | | | |
| IBA MANHATTAN | 1.142575 | 100000 | 0.142585 |
| IBA PLANTER'S PUNCH | 1.173702 | 100000 | 0.173712 |
| IBA CASINO | 1.201703 | 100000.2 | 0.201715 |
| IBA LONG ISLAND | 1.215712 | 100000.2 | 0.215724 |
| ICED TEA | | | |
| IBA OLD FASHIONED | 1.578177 | 100000 | 0.578187 |
| IBA JOHN COLLINS | 1.625937 | 100000.2 | 0.62595 |
| IBA GIN FIZZ | 2.085005 | 100000.2 | 1.085017 |
| IBA BLOODY MARY | 2.189857 | 100000 | 1.189867 |
| IBA DAIQUIRI | 25.34937 | 3943.872 | 0 |
| IBA BACARDI | 25.34969 | 3943.872 | 0.000321 |
| IBA CAIPIRINHA | 25.35103 | 3943.872 | 0.001669 |
| IBA YELLOW BIRD | 25.35198 | 3943.872 | 0.002614 |
| IBA KAMIKAZE | 25.35208 | 3943.853 | 0.002594 |
| IBA MAI-TAI | 25.35575 | 3943.891 | 0.006501 |
| IBA TOMMY'S | 25.36818 | 3943.853 | 0.018698 |
| MARGARITA | | | |
| IBA MARGARITA | 25.37753 | 3943.853 | 0.028044 |
| IBA COSMOPOLITAN | 25.37822 | 3943.853 | 0.028731 |
| IBA HEMINGWAY | 25.38772 | 3943.872 | 0.038351 |
| SPECIAL | | | |
| IBA CUBA LIBRE | 26.02621 | 3943.872 | 0.676848 |
| IBA MOSCOW MULE | 26.76994 | 3943.853 | 1.420451 |
| IBA SINGAPORE SLING | 28.98634 | 3944.082 | 3.638322 |
| IBA FRENCH MARTINI | 29930.26 | 2.341106 | 0.050561 |
| IBA ANGEL FACE | 81395.41 | 0.228571 | 0.015874 |
| IBA PARADISE | 81401.53 | 0.228478 | 0.025998 |
| IBA MONKEY GLAND | 81401.54 | 0.228478 | 0.027437 |
| IBA NEGRONI | 81401.55 | 0.228478 | 0.043046 |
| IBA VESPER | 81401.56 | 0.228478 | 0.050387 |
| IBA DRY MARTINI | 81401.59 | 0.228478 | 0.0841 |
| IBA GIN & TONIC 1:2 | 81401.6 | 0.228478 | 0.088917 |
| IBA GIN & TONIC 1:1 | 81401.65 | 0.228478 | 0.140733 |
| IBA DERBY | 81401.69 | 0.228478 | 0.177046 |
| IBA GIN & TONIC 1:3 | 81401.81 | 0.228478 | 0.300603 |
| IBA TUXEDO | 81401.86 | 0.228478 | 0.349205 |
| IBA GIN & TONIC 2:3 | 81402.24 | 0.228478 | 0.72713 |

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as can be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention can contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Variations on embodiments disclosed herein will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1-43. (canceled)

44. A method comprising,
using a computer with a processor and memory,
converting physicochemical data for a target ingredient to sensory data,
wherein the sensory data comprise sensory parameters categorized according to one or more sensory modalities;
comparing the sensory parameters for a first sensory modality of the target ingredient to those of the corresponding modality of a plurality of candidate substitute ingredients to calculate a plurality of proximity values,
wherein each proximity value represents a degree of similarity between the sensory modality of the target ingredient and that of each candidate ingredient within the plurality;
identifying one or more substitute ingredients among the plurality, based on a pre-determined threshold value of proximity value;
if no additional sensory modalities after the first modality are present, providing a list of substitute ingredients ranked according to proximity values, based on results from a step;
if additional sensory modalities after the first modality are present, calculating new proximity values for each additional sensory modality and combining proximity values for the same candidate substitute ingredient to calculate a global proximity value for the candidate substitute ingredient; and
identifying one or more substitute ingredients among the plurality, based on a pre-determined threshold value of global proximity value.

45. The method of claim 44 wherein the step includes,
receiving a selection from a menu of multiple options, via an interface on a computer or mobile device, a target ingredient or a target dish;
wherein the target ingredient or target dish is received at a remote server to trigger comparison between the target ingredient or target dish and one or more candidate substitute ingredients or substitute dishes to calculate proximity values to reflect the similarities between the target ingredient or target dish and the candidate substitute ingredients and candidate substitute dishes; and
sending a list of substitute ingredients or substitute dishes to the user via the computer or mobile device.

46. The method of claim 45 wherein a dish is a food or drink product.

47. The method of claim 44 wherein the converting physicochemical data for a target ingredient to sensory data, is by simple linear transformation.

48. The method of claim 44 wherein the converting physicochemical data for a target ingredient to sensory data, is by logarithmic transformation.

49. The method of claim 44 wherein the converting physicochemical data for a target ingredient to sensory data, is by sigmoidal transformation.

50. The method of claim 44 wherein the converting physicochemical data for a target ingredient to sensory data, is by multiple types of transformation.

51. The method of claim 44 further comprising, calculating an odor activity (OAV) value for a particular molecule in a target ingredient, by dividing its concentration by its corresponding overall threshold value.

52. The method of claim 51 further comprising, converting skew values into workable OAV values by capping at a maximum, setting to zero if the value falls below a minimum, or using as an input into an exponential/logarithmic function.

53. The method of claim 51 further comprising, obtaining a starting intensity by applying a power law algorithm to the OAV values and applying a skewed sigmoidal or logarithmic fit to the power law algorithm to stimulate high-end values representing saturation of the human olfactory system.

54. A non-transitory computer-readable medium having computer-executable instructions thereon for a method, the method comprising,
using a computer with a processor and memory,
converting physicochemical data for a target ingredient to sensory data,
wherein the sensory data comprise sensory parameters categorized according to one or more sensory modalities;
comparing the sensory parameters for a first sensory modality of the target ingredient to those of the corresponding modality of a plurality of candidate substitute ingredients to calculate a plurality of proximity values,
wherein each proximity value represents a degree of similarity between the sensory modality of the target ingredient and that of each candidate ingredient within the plurality;
identifying one or more substitute ingredients among the plurality, based on a pre-determined threshold value of proximity value;
if no additional sensory modalities after the first modality are present, providing a list of substitute ingredients ranked according to proximity values, based on results from a step;
if additional sensory modalities after the first modality are present, calculating new proximity values for each additional sensory modality and combining proximity values for the same candidate substitute ingredient to calculate a global proximity value for the candidate substitute ingredient; and
identifying one or more substitute ingredients among the plurality, based on a pre-determined threshold value of global proximity value.

55. The non-transitory computer-readable medium of claim 54 wherein the step includes,
receiving a selection from a menu of multiple options, via an interface on a computer or mobile device, a target ingredient or a target dish;
wherein the target ingredient or target dish is received at a remote server to trigger comparison between the target ingredient or target dish and one or more candidate substitute ingredients or substitute dishes to calculate proximity values to reflect the similarities between the target ingredient or target dish and the candidate substitute ingredients and candidate substitute dishes; and
sending a list of substitute ingredients or substitute dishes to the user via the computer or mobile device.

56. The non-transitory computer-readable medium of claim 55 wherein a dish is a food or drink product.

57. The non-transitory computer-readable medium of claim 54 wherein the converting physicochemical data for a target ingredient to sensory data, is by simple linear transformation.

58. The non-transitory computer-readable medium of claim 54 wherein the converting physicochemical data for a target ingredient to sensory data, is by logarithmic transformation.

59. The non-transitory computer-readable medium of claim 54 wherein the converting physicochemical data for a target ingredient to sensory data, is by sigmoidal transformation.

60. The non-transitory computer-readable medium of claim 54 wherein the converting physicochemical data for a target ingredient to sensory data, is by multiple types of transformation.

61. The non-transitory computer-readable medium of claim 54 further comprising, calculating an odor activity (OAV) value for a particular molecule in a target ingredient, by dividing its concentration by its corresponding overall threshold value.

62. The non-transitory computer-readable medium of claim 61 further comprising, converting skew values into workable OAV values by capping at a maximum, setting to zero if the value falls below a minimum, or using as an input into an exponential/logarithmic function.

62. The non-transitory computer-readable medium of claim 61 further comprising, obtaining a starting intensity by applying a power law algorithm to the OAV values.

63. The non-transitory computer-readable medium of claim 62 further comprising, applying a skewed sigmoidal or logarithmic fit to the power law to stimulate high-end values representing saturation of the human olfactory system.
